SEQUENCE LISTING (21 sequences)

SEQ ID NO: 1 - DNA, 885 bp, Trichoderma reesei
  source 1..885: mol_type = unassigned DNA
atggtttggggtgctggatcgagtgaactcctggtcgaggccaacttttacttcgaagctcgcgacgcagatcaaggcggtgactacctgatgcgagctttcaagcagctcgagctggacctcagaaagctctcgccacaaggcatcatggaactaatcctgggtatgatcaatcgggatcccggcatgatgacggccctttgcaagtaccttgcagcatattcgacaaccaatctggagcgaacccatcctctccggcaaatcttcacttgtctgtatgaagtgcaacaaaagcatggggcgcagacgttgtctgagctcctgtggactagcatctcgacaattgcggaggaactcgaggccatctatggacgcaagcatccgtatgtggctcgcacatgggccgatcttgcgctgttttacagccaggtgaacccggaaaggctggagaagttggttgttgagcttcgtgtgctccagaggcagctcgagcaacgacatgggcattccagtgtcgaagtggtttccatccgatatgccattctgctgttggtctatgcgtcgtctccccagtcggatgcctcgaagcaagccgcaaatgattattggaacctgctgcggaatatgaacaccatgtttcccatgcgcgactcccgtccgaatagttactgctatcacagcccgctcaaggtcgatccgtggacaaagaggtgccgcaggcggtacgacacactcgtcaccatattcgaggagcatgtaggcgttagaatcaatccctatttcgaagaggacttccacacgaccgagcacgctcaagaaacgcaggatgcctgggcggcagctctgcaaatgggttcgacgaatagatcttggggcttcatctag

SEQ ID NO: 2 - DNA, 1344 bp, Trichoderma reesei
  source 1..1344: mol_type = unassigned DNA
atggtttggggtgctggatcgagtgaactcctggtcgaggccaacttttacttcgaagctcgcgacgcagatcaaggcggtgactacctgatgcgagctttcaagcagctcgagctggacctcagaaagctctcgccacaaggcatcatggaactaatcctgggtatgatcaatcgggatcccggcatgatgacggccctttgcaagtaccttgcagcatattcgacaaccaatctggagcgaacccatcctctccggcaaatcttcacttgtctgtatgaagtgcaacaaaagcatggggcgcagacgttgtctgagctcctgtggactagcatctcgacaattgcggaggaactcgaggccatctatggacgcaagcatccccaggtgaacccggaaaggctggagaagttggttgttgagcttcgtgtgctccagaggcagctcgagcaacgacatgggcattccagtgtcgaagtggtttccatccgatatgccattctgctgttggtctatgcgtcgtctccccagtcggatgcctcgaagcaagccgcaaatgattattggaacctgctgcggaatatgaacaccatgtttcccatgcgcgactcccgtccgaatagttactgctatcacagcccgctcaaggtcgatccgtggacaaagaggtgccgcaggcggtacgacacactcgtcaccatattcgaggagcatgtaggcgttagaatcaatccctatttcgaagaggacttccacacgaccgagcacgctcaagaaacgcaggatgcctgggcggcagctctgcaaatgggttcgacgaatagatcttggggcttcatctagcggtttcgctttcagaatgttgggtgtgttccacgcccagctggcgcaaatgtcaggtacccaagataccttgtctctcgtctcctagcgcgcggtgcagcgagcttctttttcgagatttctttttccggaatctagcgggcggttaatacccagcatcagaaggattgcggcgcatctacctagcgacagtttttttcggttacgtcctgtttgccacctcacatcgagcctggagttttggacagttgacaattctcgccatcgcggaacattttcctttctcatgtcgccgacgctgacaagtcgatttccttggtctttccacgcaagttgttgcacctctcgagccgggcgttagggagggccttttcccccttggatgcgtcgtcatccgtgtataatagtatccgttgttttctgttttcctttcctgcgtttgatatgcgatgttcagctttcttgatactgaagaccacgagggagaggcgggaaacgagt

SEQ ID NO: 3 - protein, 280 aa, Trichoderma reesei
MVWGAGSSELLVEANFYFEARDADQGGDYLMRAFKQLELDLRKLSPQGIMELILGMINRDPGMMTALCKYLAAYSTTNLERTHPLRQIFTCLYEVQQKHGAQTLSELLWTSISTIAEELEAIYGRKHPQVNPERLEKLVVELRVLQRQLEQRHGHSSVEVVSIRYAILLLVYASSPQSDASKQAANDYWNLLRNMNTMFPMRDSRPNSYCYHSPLKVDPWTKRCRRRYDTLVTIFEEHVGVRINPYFEEDFHTTEHAQETQDAWAAALQMGSTNRSWGFI

SEQ ID NO: 4 - DNA, 2749 bp, Trichoderma reesei
  source 1..2749: mol_type = unassigned DNA
atgttccatcaaccgccggaaaagcctcgcgcgcctggcttcgacgatgatccgttcgggtttgtcacccaggcctgggagagatatggcctgggcagtgtgtcgtcgcctccgcctgcgatgcctccgcagcccatccagaggaccggacgggcctattccaccgcttcggtaatggactggacgcccgagatcgataccacctttcaccaccacggcgacggagtggccatgtcgccggtggaacacgtcgaactggacagccttggttcgcctcagggagggggaggggttggccgcttggtcgcgcactttgagaataaaggctatgtcccgccattgcctccgcggcccatcaacaatcagccgcatcaaccgcatcacatgggccacagtccttcaatgtcgtcgcagtttggcagtctaaactctgtagcccatacgcataggatatcgagtcccgttgctagcccaggtgaatcccaatatggctcgctcggggtgcattcgcccccaacacccacctcaatcgtcaccaacatgaaccgctccggcagtataagctatggcagcttccatgacactcagcaggtgcatagtcccggtattggtacgccttttgggagcatggatggctttatgagttctgcccgcgtgaacagcccgatggtggcgacaccaatggcatcgacgcccatgatgcccagtcccatggccgctccgggagtgcctggtacccctggattcgagatttggcgacctcctccctcgatgactcccaaacctgaaccctctcaaatcaccaacccagccaatttcggcggctatttcagacctccagtgccgacgactcccaagccggtagtcaacacaggtaatcagttcatcttggaattcaaccccagcgccaaggcagccaaagggaaagctccggcaaagcctcccaggcctcgattgcctcctcggaaacccaacctttcacaatcacaactctcggcacaaacaccagcagcacctgtgcaagcgtcggtttcaacaccggcaccggcatcggcaccgacaccgacaccagccgttgcctcaactccaggtccaatgtctcctcctcctaaaccgcctcgcccttcagaaccgtcttcttcatccacacccattgttgtgagcaatgccgctaaaccaagagaatccttgcagctaggtgtactaacgcgcatgtctaggcacagcggcgggattcgatggctcttcgtgcccaggcaggtactaggccgtcacgggaacaggttcctgcagaggcctgggagcaatacaaatccactatccgtaccctctatctcgaggagagaaaacccttgaaagaagtcatgagtgtcatggctgaacaatatgggtttcaagcaacgtgagtcgaagattgattcctcttctgcttcatcgttcgggttcttggttcgttgtaaactaattcgacgccctttctaggccaaagatgtataaaacaaggttctctcaatggggttttgtgaagaataacacggaggaagaagtgaaacggctgttgtcgatgaagttccagcgagatgccgagggcaaagtttccgagtttgttcgaaacggcagggtggtgaacctaggtacctatttgaaacggaaaggagtgacggagtatgacctcgttgatttcgaactaccggccgaccttccagcacatatccgatgcagaacaccgacaccacctccggctctgcgatcacccgatctgctccgtgcacaggaggtagtcgttggaaatatgcgcaaggcattcctacactgtcggcaattcgagatggagactgagactcagattggttggccatcaaccatggtttggggtgctggatcgagtgaactcctggtcgaggccaacttttacttcgaagctcgcgacgcagatcaaggcggtgactacctgatgcgagctttcaagcagctcgagctggacctcagaaagctctcgccacaaggcatcatggaactaatcctgggtatgatcaatcgggatcccggcatgatgacggccctttgcaagtaccttgcagcatattcgacaaccaatctggagcgaacccatcctctccggcaaatcttcacttgtctgtatgaagtgcaacaaaagcatggggcgcagacgttgtctgagctcctgtggactagcatctcgacaattgcggaggaactcgaggccatctatggacgcaagcatccgtatgtggctcgcacatgggccgatcttgcgctgttttacagccaggtgaacccggaaaggctggagaagttggttgttgagcttcgtgtgctccagaggcagctcgagcaacgacatgggcattccagtgtcgaagtggtttccatccgatatgccattctgctgttggtctatgcgtcgtctccccagtcggatgcctcgaagcaagccgcaaatgattattggaacctgctgcggaatatgaacaccatgtttcccatgcgcgactcccgtccgaatagttactgctatcacagcccgctcaaggtcgatccgtggacaaagaggtgccgcaggcggtacgacacactcgtcaccatattcgaggagcatgtaggcgttagaatcaatccctatttcgaagaggacttccacacgaccgagcacgctcaagaaacgcaggatgcctgggcggcagctctgcaaatgggttcgacgaatagatcttggggcttcatctag

SEQ ID NO: 5 - DNA, 2478 bp, Trichoderma reesei
  source 1..2478: mol_type = unassigned DNA
atgttccatcaaccgccggaaaagcctcgcgcgcctggcttcgacgatgatccgttcgggtttgtcacccaggcctgggagagatatggcctgggcagtgtgtcgtcgcctccgcctgcgatgcctccgcagcccatccagaggaccggacgggcctattccaccgcttcggtaatggactggacgcccgagatcgataccacctttcaccaccacggcgacggagtggccatgtcgccggtggaacacgtcgaactggacagccttggttcgcctcagggagggggaggggttggccgcttggtcgcgcactttgagaataaaggctatgtcccgccattgcctccgcggcccatcaacaatcagccgcatcaaccgcatcacatgggccacagtccttcaatgtcgtcgcagtttggcagtctaaactctgtagcccatacgcataggatatcgagtcccgttgctagcccaggtgaatcccaatatggctcgctcggggtgcattcgcccccaacacccacctcaatcgtcaccaacatgaaccgctccggcagtataagctatggcagcttccatgacactcagcaggtgcatagtcccggtattggtacgccttttgggagcatggatggctttatgagttctgcccgcgtgaacagcccgatggtggcgacaccaatggcatcgacgcccatgatgcccagtcccatggccgctccgggagtgcctggtacccctggattcgagatttggcgacctcctccctcgatgactcccaaacctgaaccctctcaaatcaccaacccagccaatttcggcggctatttcagacctccagtgccgacgactcccaagccggtagtcaacacaggtaatcagttcatcttggaattcaaccccagcgccaaggcagccaaagggaaagctccggcaaagcctcccaggcctcgattgcctcctcggaaacccaacctttcacaatcacaactctcggcacaaacaccagcagcacctgtgcaagcgtcggcacagcggcgggattcgatggctcttcgtgcccaggcaggtactaggccgtcacgggaacaggttcctgcagaggcctgggagcaatacaaatccactatccgtaccctctatctcgaggagagaaaacccttgaaagaagtcatgagtgtcatggctgaacaatatgggtttcaagcaacgccaaagatgtataaaacaaggttctctcaatggggttttgtgaagaataacacggaggaagaagtgaaacggctgttgtcgatgaagttccagcgagatgccgagggcaaagtttccgagtttgttcgaaacggcagggtggtgaacctaggtacctatttgaaacggaaaggagtgacggagtatgacctcgttgatttcgaactaccggccgaccttccagcacatatccgatgcagaacaccgacaccacctccggctctgcgatcacccgatctgctccgtgcacaggaggtagtcgttggaaatatgcgcaaggcattcctacactgtcggcaattcgagatggagactgagactcagattggttggccatcaaccatggtttggggtgctggatcgagtgaactcctggtcgaggccaacttttacttcgaagctcgcgacgcagatcaaggcggtgactacctgatgcgagctttcaagcagctcgagctggacctcagaaagctctcgccacaaggcatcatggaactaatcctgggtatgatcaatcgggatcccggcatgatgacggccctttgcaagtaccttgcagcatattcgacaaccaatctggagcgaacccatcctctccggcaaatcttcacttgtctgtatgaagtgcaacaaaagcatggggcgcagacgttgtctgagctcctgtggactagcatctcgacaattgcggaggaactcgaggccatctatggacgcaagcatccgtatgtggctcgcacatgggccgatcttgcgctgttttacagccaggtgaacccggaaaggctggagaagttggttgttgagcttcgtgtgctccagaggcagctcgagcaacgacatgggcattccagtgtcgaagtggtttccatccgatatgccattctgctgttggtctatgcgtcgtctccccagtcggatgcctcgaagcaagccgcaaatgattattggaacctgctgcggaatatgaacaccatgtttcccatgcgcgactcccgtccgaatagttactgctatcacagcccgctcaaggtcgatccgtggacaaagaggtgccgcaggcggtacgacacactcgtcaccatattcgaggagcatgtaggcgttagaatcaatccctatttcgaagaggacttccacacgaccgagcacgctcaagaaacgcaggatgcctgggcggcagctctgcaaatgggttcgacgaatagatcttggggcttcatctag

SEQ ID NO: 6 - protein, 825 aa, Trichoderma reesei
MFHQPPEKPRAPGFDDDPFGFVTQAWERYGLGSVSSPPPAMPPQPIQRTGRAYSTASVMDWTPEIDTTFHHHGDGVAMSPVEHVELDSLGSPQGGGGVGRLVAHFENKGYVPPLPPRPINNQPHQPHHMGHSPSMSSQFGSLNSVAHTHRISSPVASPGESQYGSLGVHSPPTPTSIVTNMNRSGSISYGSFHDTQQVHSPGIGTPFGSMDGFMSSARVNSPMVATPMASTPMMPSPMAAPGVPGTPGFEIWRPPPSMTPKPEPSQITNPANFGGYFRPPVPTTPKPVVNTGNQFILEFNPSAKAAKGKAPAKPPRPRLPPRKPNLSQSQLSAQTPAAPVQASAQRRDSMALRAQAGTRPSREQVPAEAWEQYKSTIRTLYLEERKPLKEVMSVMAEQYGFQATPKMYKTRFSQWGFVKNNTEEEVKRLLSMKFQRDAEGKVSEFVRNGRVVNLGTYLKRKGVTEYDLVDFELPADLPAHIRCRTPTPPPALRSPDLLRAQEVVVGNMRKAFLHCRQFEMETETQIGWPSTMVWGAGSSELLVEANFYFEARDADQGGDYLMRAFKQLELDLRKLSPQGIMELILGMINRDPGMMTALCKYLAAYSTTNLERTHPLRQIFTCLYEVQQKHGAQTLSELLWTSISTIAEELEAIYGRKHPYVARTWADLALFYSQVNPERLEKLVVELRVLQRQLEQRHGHSSVEVVSIRYAILLLVYASSPQSDASKQAANDYWNLLRNMNTMFPMRDSRPNSYCYHSPLKVDPWTKRCRRRYDTLVTIFEEHVGVRINPYFEEDFHTTEHAQETQDAWAAALQMGSTNRSWGFI

SEQ ID NO: 7 - DNA, 2751 bp, Trichoderma reesei
  source 1..2751: mol_type = unassigned DNA
atgttccatcaaccgccggaaaagcctcgcgcgcctggcttcgacgatgatccgttcgggtttgtcacccaggcctgggagagatatggcctgggcagtgtgtcgtcgcctccgcctgcgatgcctccgcagcccatccagaggaccggacgggcctattccaccgcttcggtaatggactggacgcccgagatcgataccacctttcaccaccacggcgacggagtggccatgtcgccggtggaacacgtcgaactggacagccttggttcgcctcagggagggggaggggttggccgcttggtcgcgcactttgagaataaaggctatgtcccgccattgcctccgcggcccatcaacaatcagccgcatcaaccgcatcacatgggccacagtccttcaatgtcgtcgcagtttggcagtctaaactctgtagcccatacgcataggatatcgagtcccgttgctagcccaggtgaatcccaatatggctcgctcggggtgcattcgcccccaacacccacctcaatcgtcaccaacatgaaccgctccggcagtataagctatggcagcttccatgacactcagcaggtgcatagtcccggtattggtacgccttttgggagcatggatggctttatgagttctgcccgcgtgaacagcccgatggtggcgacaccaatggcatcgacgcccatgatgcccagtcccatggccgctccgggagtgcctggtacccctggattcgagatttggcgacctcctccctcgatgactcccaaacctgaaccctctcaaatcaccaacccagccaatttcggcggctatttcagacctccacagtgccgacgactcccaagccggtagtcaacacaggtaatcagttcatcttggaattcaaccccagcgccaaggcagccaaagggaaagctccggcaaagcctcccaggcctcgattgcctcctcggaaacccaacctttcacaatcacaactctcggcacaaacaccagcagcacctgtgcaagcgtcggtttcaacaccggcaccggcatcggcaccgacaccgacaccagccgttgcctcaactccaggtccaatgtctcctcctcctaaaccgcctcgcccttcagaaccgtcttcttcatccacacccattgttgtgagcaatgccgctaaaccaagagaatccttgcagctaggtgtactaacgcgcatgtctaggcacagcggcgggattcgatggctcttcgtgcccaggcaggtactaggccgtcacgggaacaggttcctgcagaggcctgggagcaatacaaatccactatccgtaccctctatctcgaggagagaaaacccttgaaagaagtcatgagtgtcatggctgaacaatatgggtttcaagcaacgtgagtcgaagattgattcctcttctgcttcatcgttcgggttcttggttcgttgtaaactaattcgacgccctttctaggccaaagatgtataaaacaaggttctctcaatggggttttgtgaagaataacacggaggaagaagtgaaacggctgttgtcgatgaagttccagcgagatgccgagggcaaagtttccgagtttgttcgaaacggcagggtggtgaacctaggtacctatttgaaacggaaaggagtgacggagtatgacctcgttgatttcgaactaccggccgaccttccagcacatatccgatgcagaacaccgacaccacctccggctctgcgatcacccgatctgctccgtgcacaggaggtagtcgttggaaatatgcgcaaggcattcctacactgtcggcaattcgagatggagactgagactcagattggttggccatcaaccatggtttggggtgctggatcgagtgaactcctggtcgaggccaacttttacttcgaagctcgcgacgcagatcaaggcggtgactacctgatgcgagctttcaagcagctcgagctggacctcagaaagctctcgccacaaggcatcatggaactaatcctgggtatgatcaatcgggatcccggcatgatgacggccctttgcaagtaccttgcagcatattcgacaaccaatctggagcgaacccatcctctccggcaaatcttcacttgtctgtatgaagtgcaacaaaagcatggggcgcagacgttgtctgagctcctgtggactagcatctcgacaattgcggaggaactcgaggccatctatggacgcaagcatccgtatgtggctcgcacatgggccgatcttgcgctgttttacagccaggtgaacccggaaaggctggagaagttggttgttgagcttcgtgtgctccagaggcagctcgagcaacgacatgggcattccagtgtcgaagtggtttccatccgatatgccattctgctgttggtctatgcgtcgtctccccagtcggatgcctcgaagcaagccgcaaatgattattggaacctgctgcggaatatgaacaccatgtttcccatgcgcgactcccgtccgaatagttactgctatcacagcccgctcaaggtcgatccgtggacaaagaggtgccgcaggcggtacgacacactcgtcaccatattcgaggagcatgtaggcgttagaatcaatccctatttcgaagaggacttccacacgaccgagcacgctcaagaaacgcaggatgcctgggcggcagctctgcaaatgggttcgacgaatagatcttggggcttcatctag

SEQ ID NO: 8 - DNA, 2749 bp, Trichoderma reesei
  source 1..2749: mol_type = unassigned DNA
atgttccatcaaccgccggaaaagcctcgcgcgcctggcttcgacgatgatccgttcgggtttgtcacccaggcctgggagagatatggcctgggcagtgtgtcgtcgcctccgcctgcgatgcctccgcagcccatccagaggaccggacgggcctattccaccgcttcggtaatggactggacgcccgagatcgataccacctttcaccaccacggcgacggagtggccatgtcgccggtggaacacgtcgaactggacagccttggttcgcctcagggagggggaggggttggccgcttggtcgcgcactttgagaataaaggctatgtcccgccattgcctccgcggcccatcaacaatcagccgcatcaaccgcatcacatgggccacagtccttcaatgtcgtcgcagtttggcagtctaaactctgtagcccatacgcataggatatcgagtcccgttgctagcccaggtgaatcccaatatggctcgctcggggtgcattcgcccccaacacccacctcaatcgtcaccaacatgaaccgctccggcagtataagctatggcagcttccatgacactcagcaggtgcatagtcccggtattggtacgccttttgggagcatggatggctttatgagttctgcccgcgtgaacagcccgatggtggcgacaccaatggcatcgacgcccatgatgcccagtcccatggccgctccgggagtgcctggtacccctggattcgagatttggcgacctcctccctcgatgactcccaaacctgaaccctctcaaatcaccaacccagccaatttcggcggctatttcagacctccagtgccgacgactcccaagccggtagtcaacacaggtaatcagttcatcttggaattcaaccccagcgccaaggcagccaaagggaaagctccggcaaagcctcccaggcctcgattgcctcctcggaaacccaacctttcacaatcacaactctcggcacaaacaccagcagcacctgtgcaagcgtcggtttcaacaccggcaccggcatcggcaccgacaccgacaccagccgttgcctcaactccaggtccaatgtctcctcctcctaaaccgcctcgcccttcagaaccgtcttcttcatccacacccattgttgtgagcaatgccgctaaaccaagagaatccttgcagctaggtgtactaacgcgcatgtctaggcatagcggcgggattcgatggctcttcgtgcccaggcaggtactaggccgtcacgggaacaggttcctgcagaggcctgggagcaatacaaatccactatccgtaccctctatctcgaggagagaaaacccttgaaagaagtcatgagtgtcatggctgaacaatatgggtttcaagcaacgtgagtcgaagattgattcctcttctgcttcatcgttcgggttcttggttcgttgtaaactaattcgacgccctttctaggccaaagatgtataaaacaaggttctctcaatggggttttgtgaagaataacacggaggaagaagtgaaacggctgttgtcgatgaagttccagcgagatgccgagggcaaagtttccgagtttgttcgaaacggcagggtggtgaacctaggtacctatttgaaacggaaaggagtgacggagtatgacctcgttgatttcgaactaccggccgaccttccagcacatatccgatgcagaacaccgacaccacctccggctctgcgatcacccgatctgctccgtgcacaggaggtagtcgttggaaatatgcgcaaggcattcctacactgtcggcaattcgagatggagactgagactcagattggttggccatcaaccatggtttggggtgctggatcgagtgaactcctggtcgaggccaacttttacttcgaagctcgcgacgcagatcaaggcggtgactacctgatgcgagctttcaagcagctcgagctggacctcagaaagctctcgccacaaggcatcatggaactaatcctgggtatgatcaatcgggatcccggcatgatgacggccctttgcaagtaccttgcagcatattcgacaaccaatctggagcgaacccatcctctccggcaaatcttcacttgtctgtatgaagtgcaacaaaagcatggggcgcagacgttgtctgagctcctgtggactagcatctcgacaattgcggaggaactcgaggccatctatggacgcaagcatccgtatgtggctcgcacatgggccgatcttgcgctgttttacagccaggtgaacccggaaaggctggagaagttggttgttgagcttcgtgtgctccagaggcagctcgagcaacgacatgggcattccagtgtcgaagtggtttccatccgatatgccattctgctgttggtctatgcgtcgtctccccagtcggatgcctcgaagcaagccgcaaatgattattggaacctgctgcggaatatgaacaccatgtttcccatgcgcgactcccgtccgaatagttactgctatcacagcccgctcaaggtcgatccgtggacaaagaggtgccgcaggcggtacgacacactcgtcaccatattcgaggagcatgtaggcgttagaatcaatccctatttcgaagaggacttccacacgaccgagcacgctcaagaaacgcaggatgcctgggcggcagctctgcaaatgggttcgacgaatagatcttggggcttcatctag

SEQ ID NO: 9 - DNA, 2748 bp, Trichoderma reesei
  source 1..2748: mol_type = unassigned DNA
atgttccatcaaccgccggaaaagcctcgcgcgcctggcttcgacgatgatccgttcgggtttgtcacccaggcctgggagagatatggcctgggcagtgtgtcgtcgcctccgcctgcgatgcctccgcagcccatccagaggaccggacgggcctattccaccgcttcggtaatggactggacgcccgagatcgataccacctttcaccaccacggcgacggagtggccatgtcgccggtggaacacgtcgaactggacagccttggttcgcctcagggagggggaggggttggccgcttggtcgcgcactttgagaataaaggctatgtcccgccattgcctccgcggcccatcaacaatcagccgcatcaaccgcatcacatgggccacagtccttcaatgtcgtcgcagtttggcagtctaaactctgtagcccatacgcataggatatcgagtcccgttgctagcccaggtgaatcccaatatggctcgctcggggtgcattcgcccccaacacccacctcaatcgtcaccaacatgaaccgctccggcagtataagctatggcagcttccatgacactcagcaggtgcatagtcccggtattggtacgccttttgggagcatggatggctttatgagttctgcccgcgtgaacagcccgatggtggcgacaccaatggcatcgacgcccatgatgcccagtcccatggccgctccgggagtgcctggtacccctggattcgagatttggcgacctcctccctcgatgactcccaaacctgaaccctctcaaatcaccaacccagccaatttcggcggctatttcagacctccagtgccgacgactcccaagccggtagtcaacacaggtaatcagttcatcttggaattcaaccccagcgccaaggcagccaaagggaaagctccggcaaagcctcccaggcctcgattgcctcctcggaacccaacctttcacaatcacaactctcggcacaaacaccagcagcacctgtgcaagcgtcggtttcaacaccggcaccggcatcggcaccgacaccgacaccagccgttgcctcaactccaggtccaatgtctcctcctcctaaaccgcctcgcccttcagaaccgtcttcttcatccacacccattgttgtgagcaatgccgctaaaccaagagaatccttgcagctaggtgtactaacgcgcatgtctaggcacagcggcgggattcgatggctcttcgtgcccaggcaggtactaggccgtcacgggaacaggttcctgcagaggcctgggagcaatacaaatccactatccgtaccctctatctcgaggagagaaaacccttgaaagaagtcatgagtgtcatggctgaacaatatgggtttcaagcaacgtgagtcgaagattgattcctcttctgcttcatcgttcgggttcttggttcgttgtaaactaattcgacgccctttctaggccaaagatgtataaaacaaggttctctcaatggggttttgtgaagaataacacggaggaagaagtgaaacggctgttgtcgatgaagttccagcgagatgccgagggcaaagtttccgagtttgttcgaaacggcagggtggtgaacctaggtacctatttgaaacggaaaggagtgacggagtatgacctcgttgatttcgaactaccggccgaccttccagcacatatccgatgcagaacaccgacaccacctccggctctgcgatcacccgatctgctccgtgcacaggaggtagtcgttggaaatatgcgcaaggcattcctacactgtcggcaattcgagatggagactgagactcagattggttggccatcaaccatggtttggggtgctggatcgagtgaactcctggtcgaggccaacttttacttcgaagctcgcgacgcagatcaaggcggtgactacctgatgcgagctttcaagcagctcgagctggacctcagaaagctctcgccacaaggcatcatggaactaatcctgggtatgatcaatcgggatcccggcatgatgacggccctttgcaagtaccttgcagcatattcgacaaccaatctggagcgaacccatcctctccggcaaatcttcacttgtctgtatgaagtgcaacaaaagcatggggcgcagacgttgtctgagctcctgtggactagcatctcgacaattgcggaggaactcgaggccatctatggacgcaagcatccgtatgtggctcgcacatgggccgatcttgcgctgttttacagccaggtgaacccggaaaggctggagaagttggttgttgagcttcgtgtgctccagaggcagctcgagcaacgacatgggcattccagtgtcgaagtggtttccatccgatatgccattctgctgttggtctatgcgtcgtctccccagtcggatgcctcgaagcaagccgcaaatgattattggaacctgctgcggaatatgaacaccatgtttcccatgcgcgactcccgtccgaatagttactgctatcacagcccgctcaaggtcgatccgtggacaaagaggtgccgcaggcggtacgacacactcgtcaccatattcgaggagcatgtaggcgttagaatcaatccctatttcgaagaggacttccacacgaccgagcacgctcaagaaacgcaggatgcctgggcggcagctctgcaaatgggttcgacgaatagatcttggggcttcatctag

SEQ ID NO: 10 - DNA, 2749 bp, Trichoderma reesei
  source 1..2749: mol_type = unassigned DNA
atgttccatcaaccgccggaaaagcctcgcgcgcctggcttcgacgatgatccgttcgggtttgtcacccaggcctgggagagatatggcctgggcagtgtgtcgtcgcctccgcctgcgatgcctccgcagcccatccagaggaccggacgggcctattccaccgcttcggtaatggactggacgcccgagatcgataccacctttcaccaccacggcgacggagtggccatgtcgccggtggaacacgtcgaactggacagccttggttcgcctcagggagggggaggggttggccgcttggtcgcgcactttgagaataaaggctatgtcccgccattgcctccgcggcccatcaacaatcagccgcatcaaccgcatcacatgggccacagtccttcaatgtcgtcgcagtttggcagtctaaactctgtagcccatacgcataggatatcgagtcccgttgctagcccaggtgaatcccaatatggctcgctcggggtgcattcgcccccaacacccacctcaatcgtcaccaacatgaaccgctccggcagtataagctatggcagcttccatgacactcagcaggtgcatagtcccggtattggtacgccttttgggagcatggatggctttatgagttctgcccgcgtgaacagcccgatggtggcgacaccaatggcatcgacgcccatgatgcccagtcccatggccgctccgggagtgcctggtacccctggattcgagatttggcgacctcctccctcgatgactcccaaacctgaaccctctcaaatcaccaacccagccaatttcggcggctatttcagacctccagtgccgacgactcccaagccggtagtcaacacaggtaatcagttcatcttggaattcaaccccagcgccaaggcagccaaagggaaagctccggcaaagcctcccaggccttgattgcctcctcggaaacccaacctttcacaatcacaactctcggcacaaacaccagcagcacctgtgcaagcgtcggtttcaacaccggcaccggcatcggcaccgacaccgacaccagccgttgcctcaactccaggtccaatgtctcctcctcctaaaccgcctcgcccttcagaaccgtcttcttcatccacacccattgttgtgagcaatgccgctaaaccaagagaatccttgcagctaggtgtactaacgcgcatgtctaggcacagcggcgggattcgatggctcttcgtgcccaggcaggtactaggccgtcacgggaacaggttcctgcagaggcctgggagcaatacaaatccactatccgtaccctctatctcgaggagagaaaacccttgaaagaagtcatgagtgtcatggctgaacaatatgggtttcaagcaacgtgagtcgaagattgattcctcttctgcttcatcgttcgggttcttggttcgttgtaaactaattcgacgccctttctaggccaaagatgtataaaacaaggttctctcaatggggttttgtgaagaataacacggaggaagaagtgaaacggctgttgtcgatgaagttccagcgagatgccgagggcaaagtttccgagtttgttcgaaacggcagggtggtgaacctaggtacctatttgaaacggaaaggagtgacggagtatgacctcgttgatttcgaactaccggccgaccttccagcacatatccgatgcagaacaccgacaccacctccggctctgcgatcacccgatctgctccgtgcacaggaggtagtcgttggaaatatgcgcaaggcattcctacactgtcggcaattcgagatggagactgagactcagattggttggccatcaaccatggtttggggtgctggatcgagtgaactcctggtcgaggccaacttttacttcgaagctcgcgacgcagatcaaggcggtgactacctgatgcgagctttcaagcagctcgagctggacctcagaaagctctcgccacaaggcatcatggaactaatcctgggtatgatcaatcgggatcccggcatgatgacggccctttgcaagtaccttgcagcatattcgacaaccaatctggagcgaacccatcctctccggcaaatcttcacttgtctgtatgaagtgcaacaaaagcatggggcgcagacgttgtctgagctcctgtggactagcatctcgacaattgcggaggaactcgaggccatctatggacgcaagcatccgtatgtggctcgcacatgggccgatcttgcgctgttttacagccaggtgaacccggaaaggctggagaagttggttgttgagcttcgtgtgctccagaggcagctcgagcaacgacatgggcattccagtgtcgaagtggtttccatccgatatgccattctgctgttggtctatgcgtcgtctccccagtcggatgcctcgaagcaagccgcaaatgattattggaacctgctgcggaatatgaacaccatgtttcccatgcgcgactcccgtccgaatagttactgctatcacagcccgctcaaggtcgatccgtggacaaagaggtgccgcaggcggtacgacacactcgtcaccatattcgaggagcatgtaggcgttagaatcaatccctatttcgaagaggacttccacacgaccgagcacgctcaagaaacgcaggatgcctgggcggcagctctgcaaatgggttcgacgaatagatcttggggcttcatctag

SEQ ID NO: 11 - DNA, 4710 bp, Trichoderma reesei
  source 1..4710: mol_type = unassigned DNA
cgttggctcgaggcaactgcgtccgtggaggcagaatgccatcatcgatcggtcccagcacagtgtccttgagcgtacgggcaccagggggttggtatcagagcgctgccattgggcgatgtgtcctggcgagtggtggcttgcgttcatatcaccttctttttctggccgatggtggcattcacacgccgacccccctggctactccagctcaatggccggcagccctacttggtttccccggcctgattatttgcttcgccatcgctgttgcaatgttcgttagccaccagcctgcattaggttacatcccctccaatccaacctctggctcaccatgctgctgtctcttgtctgctagaggcccctctctccacctgctctcgtgcctaggctgtccccgtcgttttgatcccatcgtactagcagcggcatcaggactcctagaggcaccactcgtactagcccgtagcacaccagggccagccagaacagctccgtcctggcccagcctgtattttgagcatctctggctctcttatctcagcgggggccttctaaattgacccttctcgaaagacggcacatgggatttcccttcttggcactctagacgagggaacttttgctcgctctcttcgctgcgtggcttggcttggtttcctcccacgccgctgctgatgcttttggagctggctgctcttgttgtttgtttgtccaaaccacacagcggctgttgccctatcaaggcgttttcccacatcggccgatatctgggggaacgatacggatatctgcccctagccagccagccagccagccagccagccagccagcccttgcctgtactttcgtgctgcgcccgtcccagtaggcctctccgccgccgtgattccctctctctttactatccctctacaccaggcgattatgggcacctttgcccgcctcccatacacgaccttcgtttgactgagcaagcacgctcgccactctcagagagtgatgaatcaccgcgctggagactgattattccagccgagtcctacgacttgcccaggtccgtcagttcctgacagacaagacagccggcggactgagcaccactgctctgtctgctcggtgtgtgggcacgttgacatgttccatcaaccgccggaaaagcctcgcgcgcctggcttcgacgatgatccgttcgggtttgtcacccaggcctgggagagatatggcctgggcagtgtgtcgtcgcctccgcctgcgatgcctccgcagcccatccagaggaccggacgggcctattccaccgcttcggtaatggactggacgcccgagatcgataccacctttcaccaccacggcgacggagtggccatgtcgccggtggaacacgtcgaactggacagccttggttcgcctcagggagggggaggggttggccgcttggtcgcgcactttgagaataaaggctatgtcccgccattgcctccgcggcccatcaacaatcagccgcatcaaccgcatcacatgggccacagtccttcaatgtcgtcgcagtttggcagtctaaactctgtagcccatacgcataggatatcgagtcccgttgctagcccaggtgaatcccaatatggctcgctcggggtgcattcgcccccaacacccacctcaatcgtcaccaacatgaaccgctccggcagtataagctatggcagcttccatgacactcagcaggtgcatagtcccggtattggtacgccttttgggagcatggatggctttatgagttctgcccgcgtgaacagcccgatggtggcgacaccaatggcatcgacgcccatgatgcccagtcccatggccgctccgggagtgcctggtacccctggattcgagatttggcgacctcctccctcgatgactcccaaacctgaaccctctcaaatcaccaacccagccaatttcggcggctatttcagacctccagtgccgacgactcccaagccggtagtcaacacaggtaatcagttcatcttggaattcaaccccagcgccaaggcagccaaagggaaagctccggcaaagcctcccaggcctcgattgcctcctcggaaacccaacctttcacaatcacaactctcggcacaaacaccagcagcacctgtgcaagcgtcggtttcaacaccggcaccggcatcggcaccgacaccgacaccagccgttgcctcaactccaggtccaatgtctcctcctcctaaaccgcctcgcccttcagaaccgtcttcttcatccacacccattgttgtgagcaatgccgctaaaccaagagaatccttgcagctaggtgtactaacgcgcatgtctaggcacagcggcgggattcgatggctcttcgtgcccaggcaggtactaggccgtcacgggaacaggttcctgcagaggcctgggagcaatacaaatccactatccgtaccctctatctcgaggagagaaaacccttgaaagaagtcatgagtgtcatggctgaacaatatgggtttcaagcaacgtgagtcgaagattgattcctcttctgcttcatcgttcgggttcttggttcgttgtaaactaattcgacgccctttctaggccaaagatgtataaaacaaggttctctcaatggggttttgtgaagaataacacggaggaagaagtgaaacggctgttgtcgatgaagttccagcgagatgccgagggcaaagtttccgagtttgttcgaaacggcagggtggtgaacctaggtacctatttgaaacggaaaggagtgacggagtatgacctcgttgatttcgaactaccggccgaccttccagcacatatccgatgcagaacaccgacaccacctccggctctgcgatcacccgatctgctccgtgcacaggaggtagtcgttggaaatatgcgcaaggcattcctacactgtcggcaattcgagatggagactgagactcagattggttggccatcaaccatggtttggggtgctggatcgagtgaactcctggtcgaggccaacttttacttcgaagctcgcgacgcagatcaaggcggtgactacctgatgcgagctttcaagcagctcgagctggacctcagaaagctctcgccacaaggcatcatggaactaatcctgggtatgatcaatcgggatcccggcatgatgacggccctttgcaagtaccttgcagcatattcgacaaccaatctggagcgaacccatcctctccggcaaatcttcacttgtctgtatgaagtgcaacaaaagcatggggcgcagacgttgtctgagctcctgtggactagcatctcgacaattgcggaggaactcgaggccatctatggacgcaagcatccgtatgtggctcgcacatgggccgatcttgcgctgttttacagccaggtgaacccggaaaggctggagaagttggttgttgagcttcgtgtgctccagaggcagctcgagcaacgacatgggcattccagtgtcgaagtggtttccatccgatatgccattctgctgttggtctatgcgtcgtctccccagtcggatgcctcgaagcaagccgcaaatgattattggaacctgctgcggaatatgaacaccatgtttcccatgcgcgactcccgtccgaatagttactgctatcacagcccgctcaaggtcgatccgtggacaaagaggtgccgcaggcggtacgacacactcgtcaccatattcgaggagcatgtaggcgttagaatcaatccctatttcgaagaggacttccacacgaccgagcacgctcaagaaacgcaggatgcctgggcggcagctctgcaaatgggttcgacgaatagatcttggggcttcatctagcggtttcgctttcagaatgttgggtgtgttccacgcccagctggcgcaaatgtcaggtacccaagataccttgtctctcgtctcctagcgcgcggtgcagcgagcttctttttcgagatttctttttccggaatctagcgggcggttaatacccagcatcagaaggattgcggcgcatctacctagcgacagtttttttcggttacgtcctgtttgccacctcacatcgagcctggagttttggacagttgacaattctcgccatcgcggaacattttcctttctcatgtcgccgacgctgacaagtcgatttccttggtctttccacgcaagttgttgcacctctcgagccgggcgttagggagggccttttcccccttggatgcgtcgtcatccgtgtataatagtatccgttgttttctgttttcctttcctgcgtttgatatgcgatgttcagctttcttgatactgaagaccacgagggagaggcgggaaacgagtgttttgaaagaagttggcagtcagactttttttgttacagggcgaacgaggagttgacgggaattgagcagaagaaaaaaaatacctttatcgagcatttcctgcttcatcgggaaggagaaggcaggccggagtttggatggcaggaacggacgaggcacttgtcgattgtcgtcattgcagatggttagaccagaccgaccgtccttctgggaaggagcgggagcacactgattttctcatcgcgccttcttttcgacttgacttgccgttagtataaaaaacttcaactggtattaatgaataaagacatgatgaca

SEQ ID NO: 12 - DNA, 4082 bp, Trichoderma reesei
  source 1..4082: mol_type = unassigned DNA
ccagaacagctccgtcctggcccagcctgtattttgagcatctctggctctcttatctcagcgggggccttctaaattgacccttctcgaaagacggcacatgggatttcccttcttggcactctagacgagggaacttttgctcgctctcttcgctgcgtggcttggcttggtttcctcccacgccgctgctgatgcttttggagctggctgctcttgttgtttgtttgtccaaaccacacagcggctgttgccctatcaaggcgttttcccacatcggccgatatctgggggaacgatacggatatctgcccctagccagccagccagccagccagccagccagccagcccttgcctgtactttcgtgctgcgcccgtcccagtaggcctctccgccgccgtgattccctctctctttactatccctctacaccaggcgattatgggcacctttgcccgcctcccatacacgaccttcgtttgactgagcaagcacgctcgccactctcagagagtgatgaatcaccgcgctggagactgattattccagccgagtcctacgacttgcccaggtccgtcagttcctgacagacaagacagccggcggactgagcaccactgctctgtctgctcggtgtgtgggcacgttgacatgttccatcaaccgccggaaaagcctcgcgcgcctggcttcgacgatgatccgttcgggtttgtcacccaggcctgggagagatatggcctgggcagtgtgtcgtcgcctccgcctgcgatgcctccgcagcccatccagaggaccggacgggcctattccaccgcttcggtaatggactggacgcccgagatcgataccacctttcaccaccacggcgacggagtggccatgtcgccggtggaacacgtcgaactggacagccttggttcgcctcagggagggggaggggttggccgcttggtcgcgcactttgagaataaaggctatgtcccgccattgcctccgcggcccatcaacaatcagccgcatcaaccgcatcacatgggccacagtccttcaatgtcgtcgcagtttggcagtctaaactctgtagcccatacgcataggatatcgagtcccgttgctagcccaggtgaatcccaatatggctcgctcggggtgcattcgcccccaacacccacctcaatcgtcaccaacatgaaccgctccggcagtataagctatggcagcttccatgacactcagcaggtgcatagtcccggtattggtacgccttttgggagcatggatggctttatgagttctgcccgcgtgaacagcccgatggtggcgacaccaatggcatcgacgcccatgatgcccagtcccatggccgctccgggagtgcctggtacccctggattcgagatttggcgacctcctccctcgatgactcccaaacctgaaccctctcaaatcaccaacccagccaatttcggcggctatttcagacctccagtgccgacgactcccaagccggtagtcaacacaggtaatcagttcatcttggaattcaaccccagcgccaaggcagccaaagggaaagctccggcaaagcctcccaggcctcgattgcctcctcggaaacccaacctttcacaatcacaactctcggcacaaacaccagcagcacctgtgcaagcgtcggtttcaacaccggcaccggcatcggcaccgacaccgacaccagccgttgcctcaactccaggtccaatgtctcctcctcctaaaccgcctcgcccttcagaaccgtcttcttcatccacacccattgttgcacagcggcgggattcgatggctcttcgtgcccaggcaggtactaggccgtcacgggaacaggttcctgcagaggcctgggagcaatacaaatccactatccgtaccctctatctcgaggagagaaaacccttgaaagaagtcatgagtgtcatggctgaacaatatgggtttcaagcaacgccaaagatgtataaaacaaggttctctcaatggggttttgtgaagaataacacggaggaagaagtgaaacggctgttgtcgatgaagttccagcgagatgccgagggcaaagtttccgagtttgttcgaaacggcagggtggtgaacctaggtacctatttgaaacggaaaggagtgacggagtatgacctcgttgatttcgaactaccggccgaccttccagcacatatccgatgcagaacaccgacaccacctccggctctgcgatcacccgatctgctccgtgcacaggaggtagtcgttggaaatatgcgcaaggcattcctacactgtcggcaattcgagatggagactgagactcagattggttggccatcaaccatggtttggggtgctggatcgagtgaactcctggtcgaggccaacttttacttcgaagctcgcgacgcagatcaaggcggtgactacctgatgcgagctttcaagcagctcgagctggacctcagaaagctctcgccacaaggcatcatggaactaatcctgggtatgatcaatcgggatcccggcatgatgacggccctttgcaagtaccttgcagcatattcgacaaccaatctggagcgaacccatcctctccggcaaatcttcacttgtctgtatgaagtgcaacaaaagcatggggcgcagacgttgtctgagctcctgtggactagcatctcgacaattgcggaggaactcgaggccatctatggacgcaagcatccgtatgtggctcgcacatgggccgatcttgcgctgttttacagccaggtgaacccggaaaggctggagaagttggttgttgagcttcgtgtgctccagaggcagctcgagcaacgacatgggcattccagtgtcgaagtggtttccatccgatatgccattctgctgttggtctatgcgtcgtctccccagtcggatgcctcgaagcaagccgcaaatgattattggaacctgctgcggaatatgaacaccatgtttcccatgcgcgactcccgtccgaatagttactgctatcacagcccgctcaaggtcgatccgtggacaaagaggtgccgcaggcggtacgacacactcgtcaccatattcgaggagcatgtaggcgttagaatcaatccctatttcgaagaggacttccacacgaccgagcacgctcaagaaacgcaggatgcctgggcggcagctctgcaaatgggttcgacgaatagatcttggggcttcatctagcggtttcgctttcagaatgttgggtgtgttccacgcccagctggcgcaaatgtcaggtacccaagataccttgtctctcgtctcctagcgcgcggtgcagcgagcttctttttcgagatttctttttccggaatctagcgggcggttaatacccagcatcagaaggattgcggcgcatctacctagcgacagtttttttcggttacgtcctgtttgccacctcacatcgagcctggagttttggacagttgacaattctcgccatcgcggaacattttcctttctcatgtcgccgacgctgacaagtcgatttccttggtctttccacgcaagttgttgcacctctcgagccgggcgttagggagggccttttcccccttggatgcgtcgtcatccgtgtataatagtatccgttgttttctgttttcctttcctgcgtttgatatgcgatgttcagctttcttgatactgaagaccacgagggagaggcgggaaacgagtgttttgaaagaagttggcagtcagactttttttgttacagggcgaacgaggagttgacgggaattgagcagaagaaaaaaaatacctttatcgagcatttcctgcttcatcgggaaggagaaggcaggccggagtttggatggcaggaacggacgaggcacttgtcgattgtcgtcattgcagatggttagaccagaccgaccgtccttctgggaaggagcgggagcacactgattttctcatcgcgccttcttttcgacttgacttgccgttagtataaaaaacttcaactggtattaatgaataaagacatgatgaca

SEQ ID NO: 13 - protein, 868 aa, Trichoderma reesei
MFHQPPEKPRAPGFDDDPFGFVTQAWERYGLGSVSSPPPAMPPQPIQRTGRAYSTASVMDWTPEIDTTFHHHGDGVAMSPVEHVELDSLGSPQGGGGVGRLVAHFENKGYVPPLPPRPINNQPHQPHHMGHSPSMSSQFGSLNSVAHTHRISSPVASPGESQYGSLGVHSPPTPTSIVTNMNRSGSISYGSFHDTQQVHSPGIGTPFGSMDGFMSSARVNSPMVATPMASTPMMPSPMAAPGVPGTPGFEIWRPPPSMTPKPEPSQITNPANFGGYFRPPVPTTPKPVVNTGNQFILEFNPSAKAAKGKAPAKPPRPRLPPRKPNLSQSQLSAQTPAAPVQASVSTPAPASAPTPTPAVASTPGPMSPPPKPPRPSEPSSSSTPIVAQRRDSMALRAQAGTRPSREQVPAEAWEQYKSTIRTLYLEERKPLKEVMSVMAEQYGFQATPKMYKTRFSQWGFVKNNTEEEVKRLLSMKFQRDAEGKVSEFVRNGRVVNLGTYLKRKGVTEYDLVDFELPADLPAHIRCRTPTPPPALRSPDLLRAQEVVVGNMRKAFLHCRQFEMETETQIGWPSTMVWGAGSSELLVEANFYFEARDADQGGDYLMRAFKQLELDLRKLSPQGIMELILGMINRDPGMMTALCKYLAAYSTTNLERTHPLRQIFTCLYEVQQKHGAQTLSELLWTSISTIAEELEAIYGRKHPYVARTWADLALFYSQVNPERLEKLVVELRVLQRQLEQRHGHSSVEVVSIRYAILLLVYASSPQSDASKQAANDYWNLLRNMNTMFPMRDSRPNSYCYHSPLKVDPWTKRCRRRYDTLVTIFEEHVGVRINPYFEEDFHTTEHAQETQDAWAAALQMGSTNRSWGFI

SEQ ID NO: 14 - protein, 288 aa, Trichoderma reesei
MFHQPPEKPRAPGFDDDPFGFVTQAWERYGLGSVSSPPPAMPPQPIQRTGRAYSTASVMDWTPEIDTTFHHHGDGVAMSPVEHVELDSLGSPQGGGGVGRLVAHFENKGYVPPLPPRPINNQPHQPHHMGHSPSMSSQFGSLNSVAHTHRISSPVASPGESQYGSLGVHSPPTPTSIVTNMNRSGSISYGSFHDTQQVHSPGIGTPFGSMDGFMSSARVNSPMVATPMASTPMMPSPMAAPGVPGTPGFEIWRPPPSMTPKPEPSQITNPANFGGYFRPPQCRRLPSR

SEQ ID NO: 15 - protein, 387 aa, Trichoderma reesei
MFHQPPEKPRAPGFDDDPFGFVTQAWERYGLGSVSSPPPAMPPQPIQRTGRAYSTASVMDWTPEIDTTFHHHGDGVAMSPVEHVELDSLGSPQGGGGVGRLVAHFENKGYVPPLPPRPINNQPHQPHHMGHSPSMSSQFGSLNSVAHTHRISSPVASPGESQYGSLGVHSPPTPTSIVTNMNRSGSISYGSFHDTQQVHSPGIGTPFGSMDGFMSSARVNSPMVATPMASTPMMPSPMAAPGVPGTPGFEIWRPPPSMTPKPEPSQITNPANFGGYFRPPVPTTPKPVVNTGNQFILEFNPSAKAAKGKAPAKPPRPRLPPRKPNLSQSQLSAQTPAAPVQASVSTPAPASAPTPTPAVASTPGPMSPPPKPPRPSEPSSSSTPIVA

SEQ ID NO: 16 - protein, 430 aa, Trichoderma reesei
MFHQPPEKPRAPGFDDDPFGFVTQAWERYGLGSVSSPPPAMPPQPIQRTGRAYSTASVMDWTPEIDTTFHHHGDGVAMSPVEHVELDSLGSPQGGGGVGRLVAHFENKGYVPPLPPRPINNQPHQPHHMGHSPSMSSQFGSLNSVAHTHRISSPVASPGESQYGSLGVHSPPTPTSIVTNMNRSGSISYGSFHDTQQVHSPGIGTPFGSMDGFMSSARVNSPMVATPMASTPMMPSPMAAPGVPGTPGFEIWRPPPSMTPKPEPSQITNPANFGGYFRPPVPTTPKPVVNTGNQFILEFNPSAKAAKGKAPAKPPRPRLPPRNPTFHNHNSRHKHQQHLCKRRFQHRHRHRHRHRHQPLPQLQVQCLLLLNRLALQNRLLHPHPLLHSGGIRWLFVPRQVLGRHGNRFLQRPGSNTNPLSVPSISRRENP

SEQ ID NO: 17 - protein, 317 aa, Trichoderma reesei
MFHQPPEKPRAPGFDDDPFGFVTQAWERYGLGSVSSPPPAMPPQPIQRTGRAYSTASVMDWTPEIDTTFHHHGDGVAMSPVEHVELDSLGSPQGGGGVGRLVAHFENKGYVPPLPPRPINNQPHQPHHMGHSPSMSSQFGSLNSVAHTHRISSPVASPGESQYGSLGVHSPPTPTSIVTNMNRSGSISYGSFHDTQQVHSPGIGTPFGSMDGFMSSARVNSPMVATPMASTPMMPSPMAAPGVPGTPGFEIWRPPPSMTPKPEPSQITNPANFGGYFRPPVPTTPKPVVNTGNQFILEFNPSAKAAKGKAPAKPPRP

SEQ ID NO: 18 - protein, 297 aa, Trichoderma reesei
MFHQPPEKPRAPGFDDDPFGFVTQAWERYGLGSVSSPPPAMPPQPIQRTGRAYSTASVMDWTPEIDTTFHHHGDGVAMSPVEHVELDSLGSPQGGGGVGRLVAHFENKGYVPPLPPRPINNQPHQPHHMGHSPSMSSQFGSLNSVAHTHRISSPVASPGESQYGSLGVHSPPTPTSIVTNMNRSGSISYGSFHDTQQVHSPGIGTPFGSMDGFMSSARVNSPMVATPMASTPMMPSPMAAPGVPGTPGFEIWRPPPSMTPKPEPSQITNPANFGGYFRPPVPTTPKPVVNTGNQFIL

SEQ ID NO: 19 - DNA, 20 bp, synthetic construct
  misc_feature 1..20: note = primer
  source 1..20: mol_type = other DNA
cgttggctcgaggcaactgc

SEQ ID NO: 20 - DNA, 20 bp, synthetic construct
  misc_feature 1..20: note = primer
  source 1..20: mol_type = other DNA
tgtcatcatgtctttattca

SEQ ID NO: 21 - DNA, 20 bp, synthetic construct
  misc_feature 1..20: note = primer
  source 1..20: mol_type = other DNA
ccagaacagctccgtcctgg

(19) United States
(12) Patent Application Publication
(10) Pub. No.: US 2024/0309419 A1
(43) Pub. Date: Sep. 19, 2024

(54) METHODS FOR CONTROLLING PROTEASE PRODUCTION

(71) Applicant: ROAL OY, Rajamäki (FI)

(72) Inventors: Marja PALOHEIMO, Rajamäki (FI); Susanna MÄKINEN, Rajamäki (FI); Peter PUNT, Zeist (NL); Kari JUNTUNEN, Rajamäki (FI); Terhi PURANEN, Rajamäki (FI); Jari VEHMAANPERÄ, Rajamäki (FI)

(73) Assignee: ROAL OY, Rajamäki (FI)

(21) Appl. No.: 18/672,093

(22) Filed: May 23, 2024

Related U.S. Application Data
(63) Continuation of application No. 17/116,353, filed on Dec. 9, 2020, now Pat. No. 12,031,168.
Application No. 17/116,353 is a continuation of application No. 15/552,387, filed on Aug. 21, 2017, now Pat. No. 10,883,131.

(30) Foreign Application Priority Data
Feb. 20, 2015 (FI) 20155112

(51) Int. Cl. C12P 21/02; C07K 14/37; C07K 16/14; C12N 9/02; C12N 9/24; C12N 9/42; C12N 9/48; C12N 15/80

(52) U.S. Cl. CPC C12P 21/02; C07K 14/37; C07K 16/14; C12N 9/0061; C12N 9/2405; C12N 9/2437; C12N 9/48; C12N 15/80; C12Y 110/03002; C12Y 302/01004

(57) ABSTRACT

The present description is related to the field of protein production. It introduces novel host cells with low protease activity, a novel protease regulator, its use in expression systems and protein production, and a method of producing host cells for protein production.

### INCORPORATION BY REFERENCE

The sequence listing in XML Document (.xml) format (ST.26) is incorporated herein by reference in its entirety.
Name of File: 31256US-wbc2-SEQ_LISTING_ST26.xml
b. Date of Creation: May 20, 2024
c. Size of File: 51,235 bytes

### FIELD

The present description is related to the field protein production. More specifically, it discloses a novel protease expression regulator and its use in the production of proteins of interest in host cells.

### BACKGROUND

Microorganisms, such as fungi and filamentous fungi, are widely used as host cells for expression and extracellular secretion of proteins of interest, such as recombinant proteins. One disadvantage frequently encountered with microorganisms, when used as host cells, is their inherent production and secretion of proteolytic enzymes that degrade the protein of interest. This problem is particularly difficult when producing proteins of interest that are sensitive, unstable, or both. Thus, endogenous proteases of the host cell at least reduce the yield of the protein of interest and may even prevent its production. Additionally, proteolytic activity of the endogenous proteases may lead into formation of fragmented or degraded proteins, which lowers the quality of proteins produced in host cells. Protein authenticity may be affected by proteolysis due to trimming of N and/or C terminal amino acids by exopeptidases. Further, the presence of endogenous proteases decreases the stability and shelf life of protein compositions when the endogenous proteases are present in the protein compositions. In case longer shelf-life or stability of protein composition is desired the endogenous proteases have to be removed from the protein composition or their protease activity has to be inhibited, e.g. by protease inhibitors.

Various solutions to circumvent the above problems have been envisaged. For example, one could delete or disrupt genes encoding the various endogenous proteases, if the proteases are properly identified and characterised. WO 90/00192 describes mutant filamentous fungal hosts which have been rendered incapable of secreting an enzymatically active aspartic protease. By such mutation, it was shown that the yield of the heterologous polypeptide, bovine chymosin, could be increased. WO2013/102674 describes filamentous fungal cells that are deficient in at least three endogenous proteases, and wherein the endogenous proteases are inactivated by a mutation at the genes encoding the endogenous proteases. Attempts have also been made to inactivate endogenous proteases by random mutagenesis, but they may lead to unknown and unwanted pleiotropic effects on fermentation performance, such as problems in gene expression and poor growth rate of the host cell. Random mutagenesis produces mutations non-specifically throughout the genome of the host cell. The mutated genes producing desirable or undesirable characteristics for the host cell cannot be easily identified. The resulting mutant strains have to be used as such, even though some of the mutations might lead to non-desired outcome regarding the characteristics of the strain and/or its products.

Another approach to prevent problems of endogenous proteases has been to optimize raw materials and cultivation conditions in such a way that endogenous protease production is reduced or prevented.

However, it is well known that fungi produce a large number of endogenous proteases. Thus, strain tailoring by individually inactivating each endogenous protease is impractical. In addition, it has been shown that disruption of one protease gene may lead to a compensatory increase in the expression and production of another proteinase gene or genes. Consequently, there is an interest to develop for industrial use strains of filamentous fungi exhibiting no, or very low levels of, proteolytic activity originating from endogenous proteases. Further, it would be advantageous to provide methods that allow preventing production of endogenous proteases in host cells. In particular Trichoderma reesei with low endogenous protease activity would be particularly desirable because it is a suitable host cell for many recombinant proteins.

Some enzymes are exceptionally sensitive even to low amounts of proteases and they may need further modifications to remain stable in products such as in enzyme compositions. For example many proteins having a multi domain structure wherein the domains are linked by flexible linker regions, such as cellulases with a cellulose binding moiety, may be particularly susceptible to protease cleavage. Consequently, such enzymes may be difficult to develop into products with an acceptable shelf life and they often require careful engineering of the joining sequence in addition to using a low protease host and optimization of cultivation conditions.

### SUMMARY

It is an object to at least partially solve above problems of prior art. A related object is to improve production of proteins, especially such proteins which are sensitive to host proteases or are unstable when produced in a fungal expression system.

It is also an object to provide a method for regulating endogenous protease expression in micro-organisms.

Another object is to provide a protease regulator, a gene encoding it, and a vector comprising said gene.

It is another object to provide a protease regulator variant, a gene encoding it, and a vector comprising said gene.

It is another object to provide a method of producing a protein of interest in a host cell.

It is yet another object to provide an alternative polynucleotide and a polypeptide which regulates endogenous protease expression in a host cell.

The present inventors have surprisingly found that endogenous expression of several proteases can be suppressed in a host cell by inactivating a gene encoding a protease regulator named pea1 by the present inventors. Suppression of endogenous proteases by preventing action of pea1 in a host cell resulted into e.g. improved yield and stability of recombinant proteins produced in the host cell.

According to the first aspect of the invention there is provided a polynucleotide comprising a nucleotide sequence encoding a protein comprising an amino acid sequence having at least 90% sequence identity to amino acids 402-533 of SEQ ID NO: 13, wherein inactivation of a chromosomal gene comprising the polynucleotide results into suppression of production of endogenous proteases of the host cell compared to a host cell wherein the chromosomal gene comprising the polynucleotide is not inactivated.

The polynucleotide of the first aspect has been shown by the inventors to be responsible for producing a gene product which regulates expression of many fungal endogenous proteases. Thus, the gene is herein called a protease regulator, protease expression affecting 1, or pea1 and it is characterised at least by the presence of the sequence encoding the highly conserved region residues 402-533 of SEQ ID NO:13. The corresponding pea1 gene product (when a polypeptide) is herein called Pea1. Inhibiting the pea1 resulted in lowered levels of endogenous protease expression, as shown in Examples below. By repressing, down-regulating, inactivating or inhibiting pea1 expression it was shown to be possible to suppress, i.e. to down-regulate, expression of several endogenous proteases of the fungal host cell. The dramatic decrease in the endogenous protease activity resulted into lower degradation of proteins expressed by the host cell and, consequently, increased yield of proteins of interest, such as heterologous recombinant proteins produced by the host cell. A further advantage may be that less inactive or fragmented protein of interest may be produced because fewer endogenous proteases are produced and secreted. The protein of interest produced by the host cell may also be less prone to degradation which leads into improved authenticity, stability and shelf-life. Variants, fragments, and nucleotides that are hybridisable can be used e.g. to detect presence of the protease regulator or a sequence similar to it. The polynucleotide according to the first aspect and the gene product encoded by it are useful in industrial production of proteins.

According to the second aspect there is provided a fragment or a variant of the polynucleotide of the first aspect.

According to the third aspect there is provided a modified polynucleotide comprising the polynucleotide of the first aspect and containing at least one modification resulting into incapability of a gene product obtainable by transcribing and/or translating a chromosomal gene comprising the modified polynucleotide to induce expression of endogenous proteases in a host cell.

The modified polynucleotide of the third aspect encodes an inactive form or a fragment of the protease regulator encoded by the polynucleotide of the first aspect. It can be used to inactivate normal function of the protease regulator and, consequently, suppress endogenous protease expression in a host cell.

According to the fourth aspect there is provided a vector comprising the polynucleotide of the first aspect or the fragment or variant of the second aspect or the modified polynucleotide of the third aspect.

The polynucleotide can be inserted into the genome of a host cell for example in a vector. In certain embodiments the polynucleotide may encode an active or an inactive form of Pea1 and it may comprise genetic elements necessary for inserting the isolated polynucleotide at the region of the genome (locus) encoding the active protein by double cross-over or replacement recombination. Thus, such a polynucleotide can be used in a method for activating or inactivating the gene encoding the protease regulator of the first aspect. In an embodiment the vector is a plasmid or a phage vector. Said polynucleotides and vectors may comprise 5′ and 3′ untranslated regions, regulatory sequences of pea1 for incorporating the genetic construction into the host genome and optionally at least one marker.

According to the fifth aspect there is provided a host cell comprising at least one inactivated chromosomal gene wherein the inactivated chromosomal gene comprises a nucleic acid sequence encoding a polypeptide comprising a sequence having at least 90% sequence identity with the amino acids 402-533 of SEQ ID NO: 13.

The host cell of the fifth aspect may produce less endogenous proteases than it would normally do when the chromosomal gene is active, or not inactivated. Thus, the protein degrading activity of the endogenous proteases of the host cells can be at least partially prevented in the host cell of the fifth aspect.

According to the sixth aspect there is provided a protein preparation comprising protein produced in the host cell of the fifth aspect. In certain embodiments the protein preparation comprises host cells according to the fifth aspect.

The protein preparation may have a higher content of the protein than a corresponding protein preparation produced using the same host cell with an intact pea1. Thus, when the protein preparation is used, a smaller total volumetric amount of the protein preparation may be required to obtain the same effect that would be required when using a protein preparation produced correspondingly but in which the biological effect of the protease regulator is the same than that of a native protease regulator. Further, the authenticity, stability and the shelf life of the protein preparation may be improved when the protein preparation contains less endogenous proteases of the host cell.

According to the seventh aspect there is provided a use of the protein preparation of the sixth aspect for biomass processing or in the industry of biofuel, starch, textile, detergent, pulp and paper, food, baking, feed, beverage or pharmaceutical industry.

The use of the seventh aspect is advantageous in that as the protein preparation comprises more protein, more protein activity can be obtained from a given amount of the protein preparation and the total amount of the protein preparation used can be decreased. Also problems related to endogenous protease activity in said industrial processes may be avoided.

According to an eighth aspect there is provided a method of producing a protein comprising
a. growing the host cell of the fifth aspect in conditions suitable for producing the protein; and optionally
b. recovering the protein.

The method of the eighth aspect provides improved yield and stability of the protein. Further, the method allows producing proteins that are difficult or in some cases even impossible to produce in a host cell because of their sensitivity to endogenous proteases of the host cell. In certain embodiments the protein is a recombinant protein.

According to the ninth aspect there is provided a composition comprising at least one of: the protein preparation of the sixth aspect; and the protein obtainable by the method of the eighth aspect. In certain aspects the composition may comprise at least one additional constituent such as buffer, salt, solvent, water or detergent.

The composition is advantageous in that it may have a higher content of the protein compared to a composition produced accordingly, but in a host cell with an active pea1 capable of inducing expression of endogenous proteases. Further, the composition may have a low content of endogenous proteases. In certain embodiments the protein may be sensitive to protease degradation and obtaining a stable composition produced in a host cell with an active pea1 would require purification steps to remove endogenous proteases induced by pea1. In such a case the composition may be easier to obtain with the method of the eighth aspect, because the initial level of endogenous proteases is low. Also, the composition may have improved shelf life and stability.

According to the tenth aspect there is provided a method for making a host cell for protein production comprising suppressing endogenous protease gene expression in a host cell by at least partially inhibiting transcription or translation of the polynucleotide of the first aspect.

The method is advantageous because it can be used to suppress many endogenous proteases simultaneously. The resulting host cell may be used to produce higher yields of any protein, such as endogenous proteins, recombinant proteins, heterologous proteins or any protein produced and optionally secreted by the host cell. Non-limiting examples of types of proteases the expression of which can be at least partially suppressed are listed in Table 2. In certain embodiment the method provides a host cell which has reduced expression level of at least one protease.

According to the eleventh aspect there is provided a host cell obtainable using the method of the tenth aspect.

According to the twelfth aspect there is provided a protease regulator selected from the group consisting of
a) a polypeptide or a gene product encoded by the coding sequence of the polynucleotide of the first or the second aspect;
b) a polypeptide or a gene product encoded by the coding sequence of the polynucleotide of the third aspect;
c) a polypeptide encoded by the SEQ ID NO: 11 or 12;
d) a polypeptide comprising an amino acid sequence which has at least 90% sequence identity to amino acids 402-533 of SEQ ID NO: 13; and
e) a variant or a fragment of a polypeptide or a gene product of any one of a) to d).

The protease regulator of the twelfth aspect can be provided in a host cell to induce or suppress endogenous protease expression: a protease regulator having a biological effect of a native protease regulator may induce endogenous protease expression whereas an inactivated protease regulator may suppress endogenous protease expression. Further, fragments and variants may be used to interact with binding partners of the native pea1 gene product, e.g. to bind in a host cell an inactive fragment or variant of a pea1 gene product to a natural binding partner of a pea1 gene product.

According to the thirteenth aspect there is provided an antibody having binding specificity to the protease regulator of the twelfth aspect.

The antibody can be produced by methods known in the art. The antibody can be used to specifically bind the protease regulator. Thus, the presence of the protease regulator can be detected e.g. in an immunoassay when the antibody is directly or indirectly linked to a detectable label. Alternatively, the antibody can be used to bind the protease regulator to prevent binding of a binding partner to the protease regulator. In a further embodiment, when an antibody is used which binds a part of the protease regulator which does not participate in binding with its binding partner, the protease regulator with its binding partner can be bound in a complex with the antibody, and the binding partner can be identified with methods known in the art of protein chemistry. Thus, in an embodiment the antibody can be used as a research tool to identify biomolecules participating in regulation of protease expression.

According to the fourteenth aspect there is provided a method of inducing protease expression in a host cell by providing the protease regulator of the item a), c), d) or item e) referring to item a), c) or d) of the twelfth aspect inside or in contact with the host cell. In certain embodiments the method may comprise expressing the protease regulator in the host cell under control of promoter.

Embodiments of the present disclosure provide certain benefits. Depending on the embodiment, one or several of the following benefits may be achieved: improved protein production, possibility to produce proteins that are sensitive to proteases or otherwise unstable, improved authenticity, stability and shelf-life of compositions, decreased chemical consumption, decreased need for stabilizing agents, and decreased amounts of chemical, water and energy consumption when used in industrial processes.

### Sequence Listings

SEQ ID NO: 1: Nucleotide sequence of the QM6a genome v2.0 gene ID: 123125
SEQ ID NO: 2: Nucleotide sequence of the QM6a genome v2.0 ID: 123125 cDNA
SEQ ID NO: 3: Amino acid sequence of the QM6a genome v2.0 ID: 123125
SEQ ID NO: 4: Nucleotide sequence of the RutC-30 genome v1.0 gene ID: 85889
SEQ ID NO: 5: Nucleotide sequence of the RutC-30 genome v1.0 ID: 85889 cDNA
SEQ ID NO: 6: Amino acid sequence of the RutC-30 genome v1.0 ID:85889
SEQ ID NO: 7: Nucleotide sequence of the pea1 gene in strain 33SP #9
SEQ ID NO: 8: Nucleotide sequence of the pea1 gene in strain 31SP #4
SEQ ID NO: 9: Nucleotide sequence of the pea1 gene in strain 31UV #22
SEQ ID NO: 10: Nucleotide sequence of the pea1 gene in strain A21
SEQ ID NO: 11: Nucleotide sequence of the pea1 gene cloned from QM6a (including 1140 bp upstream and 821 bp downstream sequences)
SEQ ID NO: 12: Nucleotide sequence of the pea1 cDNA determined from QM6a (including 654 bp 5′UTR and 821 bp 3′-UTR sequences)
SEQ ID NO: 13: The deduced amino acid sequence of the full-length Pea1 protein
SEQ ID NO: 14: The deduced amino acid sequence of the Pea1 protein in strain 33SP #9
SEQ ID NO: 15: The deduced amino acid sequence of the Pea1 protein in strain 31SP #4
SEQ ID NO: 16: The deduced amino acid sequence of the Pea1 protein in strain 31UV #22
SEQ ID NO: 17: The deduced amino acid sequence of the Pea1 protein in strain A21
SEQ ID NO: 18: The truncated Pea1 protein encoded by pALK4106
SEQ ID NO: 19: primer S-ppea1
SEQ ID NO: 20: primer AS-3UTRout16
SEQ ID NO: 21: primer S-5UTR26
SEQ ID NO: 22: Fusarium oxysporum FOVG_08585
SEQ ID NO: 23: Gibberella fujikuroi FFUJ_12153
SEQ ID NO: 24: Stachybotrys chartarum S40293_07230
SEQ ID NO: 25: Claviceps purpurea CPUR_05697
SEQ ID NO: 26: Ophiocordyceps sinensis OCS_06053
SEQ ID NO: 27: Nectria haematococca NECHADRAFT_85885SEQ ID NO: 28: Metarhizium acridum MAC_08836
SEQ ID NO: 29: Villosiclava virens UV8b_6262
SEQ ID NO: 30: Acremonium chrysogenum ACRE_079620

### BRIEF DESCRIPTION OF THE FIGURES

FIG. 1, panel A shows SDS-PAGE analysis of culture supernatants from shake flask cultivations of transformants producing the 20K+CBD protein. Lanes 1-3, samples deriving from the culture of a non-low-protease host of the same strain lineage as the transformation host after 3, 5 and 7 days of cultivation, respectively; 4-6, samples from 33SP #9 pALK1769 transformants #2, #6 and #7, respectively. Equal amounts of the culture supernatants were loaded on each lane.

FIG. 1, panel B shows SDS-PAGE analysis of culture supernatants from bioreactor batch cultivations of transformants producing the 20K+CBD protein from the pALK1769 expression cassette. Lane 1, sample deriving from the culture of a non-low-protease host of the same strain lineage as the transformation hosts; 2-6, samples from the cultures of strains transformed with pALK1769; one 33UV #82 transformant, two parallel 33SP #9 transformants, one 33UV #48 and one 33SP #11 transformant, respectively. Samples were taken after four days of cultivation in bioreactors. Equal amounts of the culture supernatants were loaded on each lane.

FIG. 2 schematically shows the annotations of the QM_ID123125, Rut_ID85889 and the annotation deduced from the cDNA derived from QM6a (pea1). The location of the mutations in strains 33SP #9, 31SP #4, 31UV #22 and A21 are shown with triangles in the pea1 annotation scheme.

FIG. 3 shows the nucleotide sequence of the pea1 gene (nucleotides 1141-3889 from SEQ ID NO: 11) and the deduced amino acid sequence. The length and location of the introns was determined from cDNA analysis and are shown in underlined, italics letters.

FIG. 4 shows the alignment of the amino acids of the Pea1 highly conserved region (amino acids 402-533 from SEQ ID NO: 13) with the corresponding regions of similar sequences from multiple species. Below the alignment is a symbol representing identical residues (*), conservative residues (:) and non-conservative residues ( ) according to a sequence alignment performed with Clustal Omega (https://www.ebi.ac.uk/Tools/msa/clustalo/).

FIG. 5A shows the pALK4104 cassette for full-length pea1 gene deletion, the 6748 bp EcoRI—PstI fragment cleaved from the plasmid pALK4104. A selection of restriction enzyme sites is shown. pea1_5′ and pea1_3′, 5′- and 3′-flanking regions of the pea1 gene, respectively, used for targeting the deletion cassette to the pea1 locus for pea1 gene replacement with the marker gene; syn-amdS, synthetic amdS gene encoding acetamidase for selection of transformants; Rut_ID120107 and Rut_ID10852, the location and ID numbers of annotated genes according to RutC-30 public genome sequence; QM_ID66437, the location and ID number of an annotated gene, according to QM6a public genome sequence.

FIG. 5B shows the pALK4106 cassette for pea1 truncation, the 6595 bp EcoRI—PstI fragment cleaved from the plasmid pALK4106. A selection of restriction enzyme sites is shown. pea1′, a truncated pea1 gene; pea1_3′, syn-amdS, Rut_ID120107, Rut_ID10852 and QM_ID66437; identical genes/regions to those described for pALK4104 cassette (FIG. 5A).

FIG. 6 shows the pALK4107 cassette for full-length pea1 gene deletion using the ble marker gene, the 7615 bp EcoRI—PstI fragment cleaved from the plasmid pALK4107. A selection of restriction enzyme sites is shown. pea_5′, pea_3′, Rut_ID120107, Rut_ID10852 and QM_ID66437, identical to those described for pALK4104 cassette (FIG. 5A); ble, gene originating from Streptoalloteichus hindustanus and encoding ShBle, giving resistance to antibiotics of the phleomycin family; pgpdA and ttrpC, originating from Aspergillus nidulans, the promoter from glyceraldehyde-3-phosphate dehydrogenase gene and terminator from a gene encoding polypeptide acting in the tryptophan biosynthesis, respectively. The ble with promoter and terminator were isolated from pAN8-1 plasmid (Mattern et al., 1988; NCBI gi: 475899).

FIG. 7 shows a sequence alignment of the deduced amino acid sequences of the truncated Pea1 proteins in strains 33SP #9 (SEQ ID NO: 14), 31SP #4 (SEQ ID NO: 15), 31UV #22 (SEQ ID NO: 16) and A21 (SEQ ID NO: 17) and the deduced amino acid sequence of the truncated Pea1 protein (SEQ ID NO: 18) encoded by the truncated pea1 in pALK4106 (FIG. 5B). The amino acids not matching to the amino acid sequence of the native Pea1 (SEQ ID NO: 13), i.e. amino acids generated by a frame-shift, are underlined.

FIG. 8. SDS-PAGE analysis of Δpea1 transformants and host producing a recombinant cellulase protein. Samples were run into 12% SDS-polyacrylamide gel from culture supernatants of laboratory scale fermentations run for four days (same amount of sample from each fermentation). The gel was stained with Coomassie Blue. 1, molecular mass marker; 2, culture supernatant from RF5969 cultivation; 3-7, culture supernatants from cultivations of five separate RF5969 transformants with pea1 deletion.

FIG. 9. Design of split marker approach to disrupt the pea1 homologues from Fusarium species. Ppea1, promoter region of the Fusarium oxysporum pea1 gene; pea1, F. oxysporum pea1 gene; Tpea1, terminator region of the F. oxysporum pea1 gene, PgpdA, promoter of Aspergillus glyseraldehyde-3-phosphate dehydrogenase gene; hph, gene encoding hygromycin phosphotransferase (for hygromycin resistance); TtrpC, terminator of the Aspergillus trpC (tryptophan C) gene. The regions for possible homologous recombinations are shown by crosses. The fragment sizes are not in scale.

FIG. 10. Protease activities from the culture supernatants of Fusarium oxysporum, F. fujikuroi and their transformants with disrupted pea1 gene. OXY WT and OXY-03, OXY-09, OXY-24, OXY-38, the protease activity analysed from the culture supernatants of F. oxysporum Fo47 and its four transformants, respectively; FUJI WT and FUJI-01, FUJI-08, FUJI-11 and FUJI-31, the protease activity results from the culture supernatants of F. fujikuroi IMI58289 and its four transformants, respectively.

### DEPOSITS

The following strain depositions according to the Budapest Treaty on the International Recognition of Deposit of Microorganisms for the Purposes of Patent Procedure were made:

The E.coli strain RF11697 including the plasmid pALK3535 was deposited at the Deutsche Sammlung von Mikroorganismen und Zellkulturen GmbH (DSMZ), Inhoffenstrasse 7 b, D-38124 Braunschweig, Germany on 4 Feb. 2015 and assigned accession number DSM 32007.

The E.coli strain RF11698 including the plasmid pALK3536 was deposited at the Deutsche Sammlung von Mikroorganismen und Zellkulturen GmbH (DSMZ), Inhoffenstrasse 7 b, D-38124 Braunschweig, Germany on 4 Feb. 2015 and assigned accession number DSM 32008.

### DETAILED DESCRIPTION

Contrary to observations in prior art, the present inventors have identified and characterized a fungal protease expression regulator and successfully engineered a host cell suitable for industrial use which lacks the functional protease expression regulator or in which the protease regulator is inactivated. Without being bound to any theory, the present disclosure shows that by inactivating the protease regulator, expression levels of several endogenous proteases of the host cell can be significantly reduced. Thus, when the endogenous protease regulator is suppressed in a host cell, production of a protein of interest may be enhanced, resulting into improved yield and reduced proteolytic degradation of produced and/or secreted proteins. Simultaneously, fermentation performance, proliferation and protein production capabilities of the host cell may be maintained at levels required in industrial production of proteins.

As used herein, “pea1” means a polynucleotide comprising the sequence of SEQ ID NO: 11 nucleotides 1141-3889, as well as the sequence of the coding region in SEQ ID NO: 12 and sequences having similarity with said SEQ ID NOs. The pea1 gene encodes a gene product the suppression of which results into lowered expression of many fungal endogenous proteases. Thus, the gene is called a protease regulator, protease expression affecting 1, or pea1. 5′ and 3′ untranslated regions, promoter regions, introns, exons and regulatory sequences may have an effect on the function of pea1.

In certain embodiments the polynucleotide or the polypeptide of any aspect or embodiment is an isolated polynucleotide or an isolated polypeptide.

As used herein, “isolated” means a substance in a form or environment that does not occur in nature. Non-limiting examples of isolated substances include (a) any non-naturally occurring substance, (2) any substance including any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing or decreasing the amount of the substance relative to other components with which it is naturally associated (e.g., recombinant production in a host cell; multiple copies of a gene encoding the substance; and use of an alternative promoter to the promoter naturally associated with the gene encoding the substance).

As used herein, the term “comprising” includes the broader meanings of “including”, “containing”, and “comprehending”, as well as the narrower expressions “consisting of” and “consisting only of”.

As used herein, “fragment” means a protein or a polynucleotide having one or more amino acids or nucleotides deleted. In the context of DNA, a fragment includes both single stranded and double stranded DNA of any length. A fragment may be an active fragment which has the biological function, such as enzyme activity or regulatory activity, of the protein or the polynucleotide. A fragment may also be an inactive fragment, i.e. it does not have one or more biological effects of the native protein or polynucleotide.

As used herein, “variant” means a fragment of sequence (nucleotide or amino acid) inserted or deleted by one or more nucleotides/amino acids or which is chemically modified.

As used herein, a “peptide” and a “polypeptide” are amino acid sequences including a plurality of consecutive polymerized amino acid residues. For purpose of this invention, peptides are molecules including up to 20 amino acid residues, and polypeptides include more than 20 amino acid residues. The peptide or polypeptide may include modified amino acid residues, naturally occurring amino acid residues not encoded by a codon, and non-naturally occurring amino acid residues. As used herein, a “protein” may refer to a peptide or a polypeptide of any size. A protein may be an enzyme, a protein, an antibody, a membrane protein, a peptide hormone, regulator, or any other protein.

As used herein, “modification”, “modified”, and similar terms in the context of polynucleotides refer to modification in a coding or a non-coding region of the polynucleotide, such as a regulatory sequence, 5′ untranslated region, 3′ untranslated region, up-regulating genetic element, down-regulating genetic element, enhancer, suppressor, promoter, exon, or intron region. The modification may in some embodiments be only structural, having no effect on the biological effect, action or function of the polynucleotide. In other embodiments the modification is a structural modification which provides a change in the biological effect, action or function of the polynucleotide. Such a modification may enhance, suppress or change the biological function of the polynucleotide.

As used herein, “identity” means the percentage of exact matches of amino acid residues between two aligned sequences over the number of positions where there are residues present in both sequences. When one sequence has a residue with no corresponding residue in the other sequence, the alignment program allows a gap in the alignment, and that position is not counted in the denominator of the identity calculation. In this case, identity is a value determined with the Pairwise Sequence Alignment tool EMBOSS Needle at the EMBL-EBI website (www.ebi.ac.uk/Tools/psa/emboss_needle/).

As used herein, “similarity” means the percentage of matches between two sequences over the reported aligned region. In addition to identically matching amino acids (identity), similarity allows conservative substitutions (change to an amino acid with similar physical-chemical properties) to be factored into the percentage value. In this case, similarity is a value determined with the Pairwise Sequence Alignment tool EMBOSS Needle at the EMBL-EBI website (www.ebi.ac.uk/Tools/psa/emboss_needle/).

As used herein, “host cell” means any cell type that is susceptible to transformation, transfection, transduction, or the like with a nucleic acid construct or expression vector comprising a polynucleotide. The term “host cell” encompasses any progeny that is not identical due to mutations that occur during replication. Non-limiting examples of a host cell are fungal cells, filamentous fungal cells from Division Ascomycota, Subdivision Pezizomycotina; preferably from the group consisting of members of the Class Sordariomycetes, Subclass Hypocreomycetidae, Orders Hypocreales and Microascales and Aspergillus, Chrysosporium, Myceliophthora and Humicola; more preferably from the group consisting of Families Hypocreacea, Nectriaceae, Clavicipitaceae, Microascaceae, and Genera Trichoderma (anamorph of Hypocrea), Fusarium, Gibberella, Nectria, Stachybotrys, Claviceps, Metarhizium, Villosiclava, Ophiocordyceps, Cephalosporium, and Scedosporium; more preferably from the group consisting of Trichoderma reesei (Hypocrea jecorina), T. citrinoviridae, T. longibrachiatum, T. virens, T. harzianum, T. asperellum, T. atroviridae, T. parareesei, , Fusarium oxysporum, F. gramineanum, F. pseudograminearum, F. venenatum, Gibberella fujikuroi, G. moniliformis, G. zeaea, Nectria (Haematonectria) haematococca, Stachybotrys chartarum, S. chlorohalonata, Claviceps purpurea, Metarhizium acridum, M. anisopliae, Villosiclava virens, Ophiocordyceps sinensis, Acremonium (Cephalosporium) chrysogenum, and Scedosporium apiospermum, and Aspergillus niger, Aspergillus awamori, Aspergillus oryzae, Chrysosporium lucknowense, Myceliophthora thermophila, Humicola insolens, and Humicola grisea, most preferably Trichoderma reesei. In an embodiment the host cell is selected from the following group of strains obtainable from public collections: QM6a, ATCC13631; RutC-30, ATCC56765; QM9414, ATCC26921, and derivatives thereof.

As used herein, low stringency conditions mean for probes of at least 100 nucleotides in length conditions corresponding to hybridizing at prehybridisation and hybridisation at 55° C. in 5× SSC, 0.1% N-lauroylsarcosine, 0.02% SDS, 1% blocking reagent (Roche 11 096 176 001), following standard Southern blotting procedures for 12 to 24 hours. The carrier material is finally washed two to three times each for 15 minutes using 2× SSC, 0.1% SDS at 55° C.

As used herein, high stringency conditions mean for probes of at least 100 nucleotides in length conditions corresponding to hybridizing at prehybridisation and hybridization at 65° C. in 5× SSC, 0.1% N-lauroylsarcosine, 0.02% SDS, 1% blocking reagent (Roche 11 096 176 001), following standard Southern blotting procedures for 12 to 24 hours. The carrier material is finally washed two to three times each for 15 minutes using 0.1× SSC, 0.1% SDS at 65° C.

As used herein, “expression” includes any step involved in the production of a polypeptide in a host cell including, but not limited to, transcription, translation, post-translational modification, and secretion. Expression may be followed by the harvesting, i.e. recovering, the host cells or the expressed product.

As used herein, inhibiting, inactivating, suppressing and down-regulating mean at least partially preventing the biological action of pea1 gene or the gene product. As understood in the art, this can be accomplished at transcriptional, translational or protein level, i.e. by preventing reading or expressing the pea1 gene, preventing correct translation of the Pea1 protein or by preventing the pea1 gene product from binding to its binding partner(s) that in natural conditions participate in action of pea1 gene product.

As used herein a protease induced by the protease regulator of the first aspect can be any protease whose expression is induced by the protease regulator, and whose expression and/or protease activity is reduced when the protease regulator is inactivated. Non-limiting examples of such proteases are aspartic proteases, serine proteases, glutamic proteases and metalloproteases (Table 2). Thus, a biological effect of pea1 may be to regulate expression of endogenous proteases.

As used herein, a “gene product” is RNA or protein resulting from expression of a polynucleotide. Examples of gene products include mRNA, siRNA, cDNA, protein, polypeptide, and peptide.

In an example embodiment of the first aspect the host cell is Trichoderma.

In an example embodiment of the first aspect the nucleotide sequence encodes a protein comprising an amino acid sequence with 90%, 91%, 92%, 93%, 94%, 95%, 96%, 97%, 98%, 99% or 100% sequence identity to amino acids 402-533 of SEQ ID NO: 13. In another embodiment the nucleotide sequence encodes a protein comprising an amino acid sequence with 90%, 91%, 92%, 93%, 94%, 95%, 96%, 97%, 98%, 99% or 100% sequence similarity to amino acids 402-533 of SEQ ID NO: 13.

In an example embodiment of the first aspect the polynucleotide is selected from the group consisting of the coding sequence of SEQ ID NO: 11 and 12.

In an example embodiment of the first aspect the polynucleotide is selected from the group consisting of:
a) a polynucleotide comprising a sequence having at least 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with the nucleotides 1141-3889 of SEQ ID NO: 11;
b) the polynucleotide of SEQ ID NO: 12 or the coding sequence thereof;
c) the polynucleotide of SEQ ID NO: 11 or the coding sequence thereof; and
d) a nucleotide sequence hybridisable with a nucleotide sequence which is complementary to any one of a) to c) under high stringency conditions.

In an example embodiment of the first aspect the polynucleotide or its non-coding region contains at least one modification. In certain embodiments of the first aspect the modification makes it structurally different compared to any naturally occurring protease regulator, or the modification makes its expression and/or translation different, e.g. in terms of efficiency or stability compared to those of any naturally occurring protease regulator. The modification may have an effect on a biological function or another property of the protease regulator. In another embodiment the modification does not substantially change a biological function or other property of the protease regulator. Thus, in certain embodiments the modification does not substantially diminish the capability of the polynucleotide of the first aspect to induce expression of endogenous proteases in a host cell.

In an example embodiment the polynucleotide of the first aspect, the fragment or variant of the second aspect, or the modified polynucleotide of the third aspect comprises genetic elements to allow its transcription and/or translation in a host cell. In another embodiment the polynucleotide additionally comprises genetic elements that allow secreting the protein outside the host cell.

In an example embodiment of the fourth aspect the vector comprises genetic elements for incorporating the polynucleotide of the second aspect or the above embodiment into the genome of a host cell. In certain embodiments the genetic elements comprise 5′ untranslated region and/or 3′ untranslated region optionally in a form of a cassette.

In an example embodiment of the fifth aspect the host cell is selected from the group consisting of filamentous fungal cells from Division Ascomycota, Subdivision Pezizomycotina; preferably from the group consisting of members of the Class Sordariomycetes, Subclass Hypocreomycetidae, Orders Hypocreales and Microascales and Aspergillus, Chrysosporium, Myceliophthora and Humicola; more preferably from the group consisting of Families Hypocreacea, Nectriaceae, Clavicipitaceae, Microascaceae, and Genera Trichoderma (anamorph of Hypocrea), Fusarium, Gibberella, Nectria, Stachybotrys, Claviceps, Metarhizium, Villosiclava, Ophiocordyceps, Cephalosporium, and Scedosporium; more preferably from the group consisting of Trichoderma reesei (Hypocrea jecorina), T. citrinoviridae, T. longibrachiatum, T. virens, T. harzianum, T. asperellum, T. atroviridae, T. parareesei, Fusarium oxysporum, F. gramineanum, F. pseudograminearum, F. venenatum, Gibberella fujikuroi, G. moniliformis, G. zeaea, Nectria (Haematonectria) haematococca, Stachybotrys chartarum, S. chlorohalonata, Claviceps purpurea, Metarhizium acridum, M. anisopliae, Villosiclava virens, Ophiocordyceps sinensis, Acremonium (Cephalosporium) chrysogenum, and Scedosporium apiospermum, and Aspergillus niger, A. awamori, A. oryzae, Chrysosporium lucknowense, Myceliohpthora thermophila, Humicola insolens, Humicola grisea, most preferably Trichoderma reesei. In an embodiment the host cell is selected from the following group of strains obtainable from public collections: QM6a, ATCC13631; RutC-30, ATCC56765; QM9414, ATCC26921, and derivatives thereof.

In an example embodiment of the fifth aspect the inactivated chromosomal gene comprises the polynucleotide of the first aspect.

In an example embodiment of the fifth aspect the inactivated chromosomal gene is inactivated by disruption e.g. with a selectable marker, inhibition of translation or transcription of the chromosomal gene, at least partial deletion, truncation, deletion, insertion, mutation, or silencing, by RNAi or by CRISPR/Cas9 technology. When RNAi is used, double stranded RNA can be used to post-translationally silence expression levels of a specific gene, such as pea1, due to sequence-specific degradation mediated by small double-stranded RNAs. E.g. in vitro synthesised dsRNA and siRNA molecules or in vivo synthesised dsRNA or stem-loop hairpin RNA can be designed and used as triggers for targeting. When CRISPR/Cas9 technology is used in the inactivation, the Cas9 protein and appropriate guide RNAs (according to target sequence, such as pea1) are delivered into the cell, resulting to cleavage at desired location.

In an example embodiment of the fifth aspect the host cell comprises genetic elements to allow expressing, under conditions suitable for promoting expression, at least one protein of interest encoded by a recombinant polynucleotide. It is within the level of skill in the art to choose the suitable conditions, including reagents and conditions for RNA expression from the expression construct, followed by translation of the encoded polypeptide. Exemplary reagents and conditions are described in the examples that follow. The methods of this embodiment may also be carried out in a cell free translation system or in vivo. In a preferred embodiment, the protein expression is carried out in a recombinant host cell.

In an example embodiment of the fifth aspect the protein of interest is selected from the list consisting of a pharmacologically active protein, antibody, antibody fragment, therapeutic protein, biosimilar, multi-domain protein, peptide hormone, antimicrobial peptide, peptide, carbohydrate binding module, enzyme such as cellulase, protease, protease inhibitor, aminopeptidase, amylase, carbohydrase, carboxypeptidase, catalase, chitinase, cutinase, deoxyribonuclease, esterase, alpha-galactosidase, beta-galactosidase, glucoamylase, alpha-glucosidase, beta-glucosidase, invertase, laccase, lipase, mannanase, mutanase, oxidase, pectinolytic enzyme, peroxidase, phospholipase, phytase, phosphatase, polyphenoloxidase, redox enzyme, proteolytic enzyme, ribonuclease, transglutaminase and xylanase. One or more proteins of interest may be expressed by the same host cell.

In an example embodiment of the sixth aspect the protein preparation comprises at least one further component selected from stabilizer, preservative, fragrant, buffer, salt and colorant.

In an example embodiment of the tenth aspect the inhibiting is provided by making an inactivating modification in the gene comprising the sequence of the polynucleotide of the first aspect. The modification may be deletion, truncation or mutation of at least part of the protease regulator, including its control sequence, which results into suppression or at least partial inhibition of the capability of the protease regulator to induce expression of endogenous proteases in the host cell. In another embodiment the function of the protease regulator gene is inactivated post-translationally, e.g. by inhibiting protein-protein interaction or by inhibiting binding of the protease regulator to any of its natural binding partners. In yet another embodiment the protease regulator is inactivated by a deleting a promoter or other regulatory region of the present protease regulator.

In an example embodiment of the tenth aspect the inhibition is achieved by mutation, deletion, insertion, RNA interference, antibody, or small molecule inhibitor.

In an example embodiment of the eleventh aspect the host cell further comprises a nucleic acid encoding a heterologous protein.

In an example embodiment of the eleventh aspect the host cell is a fungal cell, preferably a filamentous fungal cell, such as Trichoderma or Trichoderma reesei.

### EXAMPLES

### Example 1. Isolation of Low Protease Mutants from Trichoderma reesei Strains

The Trichoderma reesei A21 is a low protease UV mutant deriving from the T. reesei QM9414 strain lineage. A21 strain was screened from the mutants obtained after ultraviolet light irradiation of parent spore batches by using a skim milk plate assay. It produced a reduced halo in the selection plate compared to its parent, indicating lowered protease production. A21 was confirmed to produce clearly lowered amounts of protease activities into its culture supernatants compared to its parent, both in shake flask cultivations and in laboratory scale bioreactors in cellulase inducing medium. It was shown by FPLC analysis that A21 lacks e.g. a protein peak which in the parent strain showed protease activity that could be inhibited by pepstatin A, indicating no or lower production of at least an aspartic type of a protease or proteases, compared to the parent.

To develop mutants with decreased production levels of native proteases from a different T. reesei mutant strain lineage, the proprietary industrial strains A31 and A33 were chosen for a strain development program. A31 is a T. reesei mutant strain with high protein (cellulase) production capacity. A33 is a genetically modified derivative from A31 from which the four major native cellulases encoding genes cbh1 (cel7A), cbh2 (cel6A), egl1 (cel7B) and egl2 (cel6A) have been deleted using the pyr4 counter selection method (for the method, see Seidl and Seiboth, 2010). The A31 and A33 mutants were generated by using UV mutagenesis and by selecting spontaneous low protease mutants using the suicide (SUI) method (Braaksma M., Punt P. J. 2008. Aspergillus as a cell factory for protein production: Controlling protease activity in fungal production. In: Goldman G H, Osmani S A, editors. The Aspergilli. Genomics, Medical Aspects, Biotechnology, and Research Methods. Boca Raton, Florida: CRC Press; Taylor & Francis) developed at TNO (The Netherlands). This method is based on a proprietary SUI chemical to which the strains producing lowered amounts of proteases are more resistant than the parent strains. By using the SUI approach the screening of low protease mutants (strains) is quick and efficient. However, the screening of such mutants can also be performed by direct plating of the mutated spores (or spores) on skim milk or other suitable protease detection plates.

The T. reesei strains were inoculated and cultivated on PD (potato dextrose agar) plates for generating spores for mutagenesis. The UV mutagenesis was conducted using BioRad UV chamber and irradiation time of 40-80 s (with survival rate of 5-50%). Non-mutagenised and UV-treated spore batches were plated on Trichoderma minimal medium (TMM; Penttilä M, H Nevalainen, M Rättö, E Salminen, and J Knowles. 1987. A versatile transformation system for the cellulolytic filamentous fungus Trichoderma reesei. Gene 61:155-164) based agar plates containing different concentrations of the SUI reagent (50-500 μg/ml) and AMM_{NH4}-plates (Bennett, J. W. and Lasure, L. L. 1991. Growth media. In More Gene Manipulations in Fungi, pp. 441-447. Edited by J. W. Bennett & L. L. Lasure. San Diego, CA: Academic Press. ISBN 0-12-088642-1) with 25-500 μg/ml of SUI to select for low protease mutants.

From both T. reesei strains about 5×10⁷ non-mutagenized and 1-2×10⁷ mutagenized spores were screened on the SUI plates. After the first SUI selection round 200-300 SUI resistant colonies from each strain were rescreened on SUI plates. About 75% of the strains still showed SUI resistant phenotype. The above type of strains were then analysed on TMM-NO₃+skim milk plates (100 ml of 10% skim milk added to TMM after autoclaving, (NH₄)₂SO₄ replaced with 6 g/l of NaNO₃). In skim milk plates about 20-40 strains (about 15% from both A31 and A33) showed no or reduced halo compared to the parental strains indicating very low or low protease production. A selection of strains was purified via single spores. These strains were further characterized on cellulose (0.5% Walseth) and xylan (0.5% oat spelt xylan) plates (A31 derived strains) or on xylan plates (A33 derived strains) to confirm that they still were capable of producing cellulase and/or xylanase activities. A selection of strains with lowered protease production, but similar cellulase and/or xylanase production on plates compared to the parents, were chosen for further analysis and characterisation. Their growth and protein and protease production levels were analysed in shake flask and bioreactor cultivations (Example 2). The suitability of chosen strains as hosts for production of protease sensitive proteins was also tested (Example 3).

### Example 2. Characterisation of the Low Protease Mutant Strains

A selection of low protease mutants, based on the plate assay results, were cultivated in shake flasks using cellulase inducing lactose based minimal medium (Bailey, M. J .; Askolin, S .; Horhammer, N .; Tenkanen, M .; Linder, M .; Penttila, M.; Nakari-Setala, T. (2002): Process technological effects of deletion and amplification of hydrophobins I and II in transformants of Trichoderma reesei. In Applied microbiology and biotechnology 58 (6), pp. 721-727). The protease activities were measured from the culture supernatants using dimethylated casein or BSA (bovine serum albumin) as substrates, based on the procedure described by Holm (Holm K.A. 1980. Automated colorimetric determination of acid proteinase activity in fermentation samples using a trinitrobenzenesulphonic acid reagent. Analyst. 105(1246): 18-24) and using glycine for calibration. For the casein assay, the pHs used in the activity measurements were 5.5, 7.0 and 8.5 and for the BSA assay pHs 4.0 and 6.0 were used. Various protease activity levels were seen in the culture supernatants among the mutant strains. However, a number of mutants (but not all) that had showed a reduced protease activity in milk halo assay also showed reduced protease activities in the liquid cultures. Some of the selected mutants showed similar or better cellulase and/or xylanase activities compared to the host. However, some of the selected mutants showed reduced cellulase and/or xylanase activities, indicating a general deficiency in protein secretion in these strains.

Based on the results from the skim milk plate assay and the minimal medium cultivation, altogether 22 A31 and 23 A33 derived low protease strains were chosen to be cultivated in shake flasks using a complex lactose-based cellulase inducing medium (Joutsjoki, V. V., T. K. Torkkeli, and K. M. H. Nevalainen. 1993. Transformation of Trichoderma reesei with the Hormoconis resinae glucoamylase P (gamP) gene: production of a heterologous glucoamylase by Trichoderma reesei. Curr. Genet. 24:223-228) buffered with 5% KH₂PO₄. The strain selection included both spontaneous and UV mutants. The protease activities as well as the amounts of secreted proteins and relevant enzyme activities (e.g. cellulase, xylanase) were quantified from the culture supernatants to confirm that the protease activities were decreased compared to the parent strain, but the amounts of other secreted proteins were not. The strains were inoculated from PD slants to shake flasks (50 ml volume of medium in 250 ml flask). Each of the strains was cultivated in two flasks with pH of the medium adjusted (prior to autoclaving the culture media) to 5.5 and 6.0. The cultivations were performed at 30° C., 250 rpm for 7 days. Samples were taken and analysed after 3, 5 and 7 days of cultivation. The pH (representing strain growth), the amount of secreted proteins (BioRad DC method), cellulase activities (hydroxyethylcellulose and 4-methylumbelliferyl-β-D-lactoside as substrates), xylanase activity (birch xylan as a substrate; Bailey, M. J .; Biely, P .; Poutanen, K. (1992): Interlaboratory testing of methods for assay of xylanase activity. In Journal of Biotechnology 23, pp. 257-270) and protease activities were measured from the culture supernatants. The protease activities were measured using haemoglobin (4.0 g in 100 ml water; at pH 4.7, 40° C., 30 min reaction; resulting to HUT activity units) and casein (1.2 g in 100 ml 30 mM ammoniumphosphate buffer; pH 7.0, 30° C., 60 min reaction) as substrates.

Some of the strains produced clearly lowered protease activities compared to their parents (Table 1). Also, a selection of the strains produced at least similar amounts of secreted proteins, cellulase and/or xylanase activities as their parent strain. Some of the strains even produced increased amounts of proteins and cellulase/xylanase activities compared to their parent. No obvious differences between the parent and the low protease strains in the protein patterns of the culture supernatants were detected in 12% SDS-PAGE gels. (Criterion XT, Biorad).

TABLE 1
Relative protease (HUT) activities measured from the culture
supernatants of the low-protease mutants grown in shake flasks for
7 days. A. A31 derived low protease mutants. B. A33 derived low
protease mutants. Results are included from a selection of
strains which produced less protease activities, but at least
similar amounts of secreted proteins and enzyme
activities as the parent strain in the cultivation. Strains
with the code NSP or SP are spontaneous mutants, those with the
code UV derive from spores treated with UV irradiation.
TMM and AMM, selection plate used (see Example
1 for details); SUI50-SUI500, concentration of the SUI reagent
on plate used in primary screening. pH 5.5 and pH 6.0, the pH
of the culture medium, adjusted prior to autoclaving.
A.

| | | Protease | |
|---|---|---|---|
| | | activity | |
| Strain | Primary | (relative HUT) | |
| No. | screening plate | pH 5.5 | pH 6.0 |
| A31 | | 100 | 100 |
| 31NSP#1 | TMM-SUI50 | 105 | 35 |
| 31SP#4 | TMM-SUI100 | 21 | 41 |
| 31SP#7 | TMM-SUI500 | 37 | 49 |
| 31UV#22 | TMM-SUI50 | 14 | 95 |
| 31NSP#6 | TMM-SUI50 | 36 | 44 |
| 31NSP#7 | TMM-SUI50 | 35 | 107 |
| 31NSP#8 | TMM-SUI50 | 62 | 42 |

B.

| | | Protease | |
|---|---|---|---|
| | | activity | |
| Strain | Primary | (relative HUT) | |
| No. | screening | pH 5.5 | pH 6.0 |
| A33 | | 100 | 100 |
| 33SP#9 | AMM-SUI25 | 29 | 43 |
| 33SP#11 | AMM-SUI100 | 27 | 44 |
| 33SP#12 | AMM-SUI150 | 26 | 34 |
| 33UV#48 | TMM-SUI50 | 26 | 50 |
| 33UV#64 | AMM-SUI50 | 27 | 39 |
| 33UV#68 | AMM-SUI50 | 30 | 37 |
| 33UV#82 | AMM-SUI50 | 37 | 45 |

A selection of A31 and A33 derived strains were cultivated in laboratory scale bioreactors in cellulase inducing complex medium. The amounts of secreted proteins, relevant enzyme activities (e.g. cellulase and xylanase activities) and protease activities were analysed from the spent culture media. The results obtained confirmed the low protease characteristics of most of the strains. The chosen samples from the fermentations were further used for analysis and identification of proteases secreted into the culture media by using protein separation, IEF and zymogram analysis and peptide mass mapping (Example 4). Samples of fungal mycelia were collected from the fermentations for Northern blot expression analysis (Example 4).

### Example 3. Low Protease Strains as Hosts for Production of Homologous and Heterologous Proteins

Chosen low protease strains deriving from A31 (31SP #4, 31UV #22 and 31NSP #6) and A33 (33SP #11, 33UV #82, 33SP #9 and 33UV #48) were tested as host for expressing two genes encoding heterologous proteins, known from previously performed expression studies to be protease sensitive when produced in T. reesei strains. The genes expressed in the chosen low protease strains were as follows: Melanocarpus albomyces derived, modified endoglucanase named as 20K+CBD (with a protease sensitive linker “WGEI”; expressed from the pALK1769 cassette; EP1874927) and Streptomyces mobaraensis transglutaminase (TGase; Washizu, K.; Ando, K.; Koikeda, S.; Hirose, S.; Matsuura, A.; Takagi, H. et al. (1994): Molecular cloning of the gene for microbial transglutaminase from Streptoverticillium and its expression in Streptomyces lividans. In Biosci. Biotechnol. Biochem. 58 (1), pp. 82-87). The genes were expressed from the native T. reesei cbh1 (cel7A) promoter. The 20K+CBD encoding gene was directly fused to the cbh1 promoter but the TGase gene (pro/mature protein encoding region) was fused 3-prime to the T. reesei Man5A carrier polypeptide encoding sequence (fused to the cbh1 promoter) in a similar way as described for a xylanase gene expression in Paloheimo et al. (Paloheimo, M., A. Mäntylä, J. Kallio, and P. Suominen. 2003. High-yield production of a bacterial xylanase in the filamentous fungus Trichoderma reesei requires a carrier polypeptide with an intact domain structure. Appl. Env. Microbiol. 69:7073-7082). The amdS (acetamidase) gene was used as a marker in both the expression cassettes.

The linear expression cassettes were isolated from the vector backbones and were transformed to protoplasts prepared from the low protease strains. The transformations were performed as in Penttilä et al. (Penttilä M, H Nevalainen, M Rättö, E Salminen, and J Knowles. 1987. A versatile transformation system for the cellulolytic filamentous fungus Trichoderma reesei. Gene 61:155-164) with the modifications described in Karhunen et al. (Karhunen, T., A. Mäntylä, K. M. H. Nevalainen, and P. L. Suominen. 1993. High frequency one step gene replacement in Trichoderma reesei. I. Endoglucanase I overproduction. Mol. Gen. Genet. 241:515-522). The transformants were purified on acetamide selection plates through single conidia prior to sporulating them on PD. The transformants were inoculated from the PD slants to shake flasks containing 50 ml of complex lactose-based cellulase inducing medium (Joutsjoki, V. V., T. K. Torkkeli, and K. M. H. Nevalainen. 1993.

Transformation of Trichoderma reesei with the Hormoconis resinae glucoamylase P (gamP) gene: production of a heterologous glucoamylase by Trichoderma reesei. Curr. Genet. 24:223-228) buffered with 5% KH₂PO₄ and PH adjusted to 5.5 or 6.0. The enzyme production of the transformants was analysed from the culture supernatants after growing them for 7 days at 30° C., 250 rpm. The chosen transformants were also cultivated in laboratory scale bioreactors using cellulase inducing complex medium and analysis of the enzyme production was performed. The production of recombinant proteins and their stability in the culture broths was analysed from the culture supernatants by enzyme activity assays and running samples on SDS-PAGE gels. For the TGase detection also a Western blot analysis was performed using in detection a commercial antibody for the bacterial transglutaminase. The stability of the recombinant protein was analysed by incubating samples of the culture supernatants at different temperatures for different periods of time and analysing them using SDS-PAGE (and/or Western blot) method.

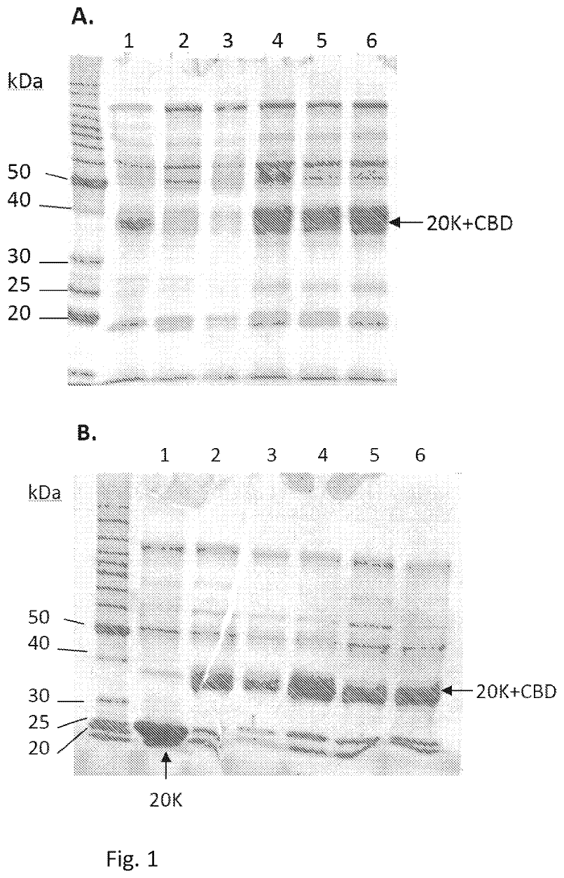

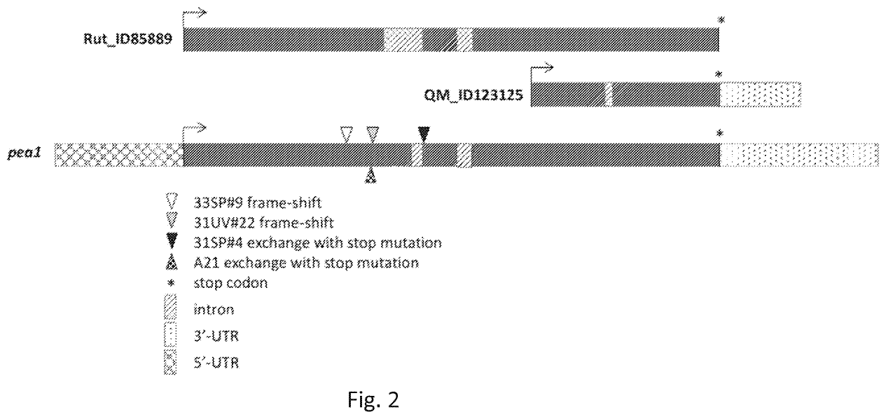

Increased amount of full-length 20K+CBD protein was produced by several of the transformants obtained, compared to the parent strain (FIG. 1A). In the low protease host the 20K+CBD protein was not degraded after 7 days of cultivation, as was shown to be the case when a host from the same strain lineage (but not a low protease mutant) was used for production of the same protein. The clearly better stability of the 20K+CBD produced in the low protease hosts was also shown in the analysis of the fermentation cultures (FIG. 1B). In these the 20K+CBD remained in the full-length form whereas the CBD was cleaved in the non-low-protease host, resulting to a 20K protein form. According to SDS-PAGE and Western blot analysis, the amounts of TGase produced by the transformants of the low protease strains were somewhat higher than the amounts produced by A31 and A33 parents. Also, the TGase produced by the low protease strains was more stable as less of the TGase degradation products were visible in fermentations samples of these strains compared to corresponding samples produced by the parent strains.

In addition to their use as hosts for heterologous proteins the low protease strains have been successfully used as hosts for homologous T. reesei proteins.

### Example 4. Proteases Produced and Expressed by the Low Protease Mutant Strains

### Identification of Proteases Not Produced or Being Less Abundant in the Low Protease Strains

Several low protease mutant strains showed highly reduced protease activities compared to their parent (Examples 1-3). A protease inhibitor study was performed to analyse in more details which type(s) of proteases were not produced or were less abundant in the culture supernatants of the low protease strains compared to their parents. Analysis of the protease activities from the culture supernatants in the absence and presence of protease inhibitors, 0.01 mM E64, 10 mM EDTA, 0.04 mg/ml Leupeptin, 1 mM Pefabloc, 0.01 mM pepstatin and 0.02 tablets/ml of Complete™ for inhibiting cysteine, divalent cation dependent, serine/cysteine, serine, aspartyl and various classes of proteases, respectively, was performed. A reduced effect of a specific inhibitor to the protease activity indicated that the mutant strain was deficient for the type of protease that is known to be inhibited by this inhibitor. The results obtained indicated that the major protease activities in the T. reesei culture supernatants were due to aspartyl and serine type of proteases. These activities were clearly reduced in the culture supernatants of several mutant strains. No inhibition of the protease activity was observed in the culture supernatants of several mutants by pepstatin (at pH 5.6), Pefabloc (at pH 4.0) or leupeptin (at pH 5.6) indicating that in these strains aspartyl and/or serine proteases were largely absent. The results obtained showed that several of the low protease strains were affected in multiple proteases. In addition to the above described protease inhibitor studies, various protein separation approaches were carried out to identify from the parents the proteases which were not produced or were less abundant in the culture supernatants of the low protease mutants. These methods included SDS-polyacrylamide gel runs, native PAGE, IEF (isoelectric focusing) gel analysis and zymogram analysis using casein-based protein gels. To reduce the background of cellulases and hemicellulases and allow better identification of the remaining protein bands, the samples for gel/IEF runs and zymogram analysis were first pre-purified (pre-absorption) using cellulose matrices. According to SDS-PAGE analysis of the non-bound protein fraction several protein bands were found to be absent in the samples deriving from the protease mutants compared to the parents. However, also new bands appeared in the samples from the cultivations of protease mutants. Differences in the patterns of secreted proteins between the samples from the parents and the low protease strains were also detected in the IEF analysis. To analyse whether the differential banding identified in the SDS-PAGE and IEF gels were proteases or corresponded to e.g. incorrectly processed proteins, or proteins which in the wildtype samples have undergone proteolytic processing of specific protein domains (e.g. CBM modules), a protease activity based zymogram analysis was carried out. At least six different protein bands with proteolytic activity could be identified using this type of analysis. The zymogram pattern of the wildtype and the mutant samples revealed several differences between these samples. For some of the protease bands it was not clear whether they were absent from the low protease strains or whether they only were less abundant.

The protein bands differing in the strains were extracted from the gels and an MS/MS analysis was performed. The protein sequence data obtainable from the Trichoderma reesei QM6a genome version 2.0 (Trire2) at http://genome.jgi-psf.org/Trire2/Trire2.home.html (ID numbers derived from this genome are hereafter referred to with a prefix QM_) was used in the identification of the proteases. In total eight different proteases were identified, four of which were clearly absent in one or more of the low protease strains.

To find additional proteases missing or being produced at lower levels by the low protease strains, also a nano-LC-MS analysis (Proxeon nLC2 and Orbitrab Elite, Thermo Fischer) was performed for the full set of proteins in the culture supernatants of several T. reesei strains, including e.g. 31UV #22. The MS data obtained was analysed using Proteome Discoverer program against the public T. reesei genome sequence (Trire2). In this analysis altogether 13 secreted T. reesei proteases were identified. Of these, at least five proteases were clearly missing or being produced in very low levels in the low protease mutant strain compared to the parent.

### Expression of Endogenous Proteases in Low Protease Strains, Northern Blot and Microarray Analysis

To analyse the expression levels of chosen protein encoding genes, RNA was isolated from samples collected from seven laboratory scale fermentations (parents and five low protease strain), from four time points (both logarithmic and stationary phases included) of the cultivations. The strains chosen for analysis were as follows: A31, A31SP #4, A31UV #22, A33, A33SP #9, A33UV #48 and A33UV #82.

The expression of the eight proteases, previously identified from the T. reesei culture supernatants, was studied. The probes were prepared by PCR, basing on sequences in the public T. reesei database. The probes were about 600 bp in length, in each case and consisted of internal fragments of the coding sequence of the 8 respective protease genes. As a reference probe, an about 600 bp gpd1 (QM_ID119735) PCR fragment was used.

The results from the Northern blot analysis showed that expression of seven out of the eight protease encoding genes was affected (no or very low expression levels) in all the mutants tested. Further transcriptional profiling of one of the low protease strains was performed using oligonucleotide microarray (Roche NimbleGen Inc., USA). Mycelia was harvested from three time points from three replicate laboratory scale fermentations of strains 33SP #9 and the wildtype strain A33 and total RNA was extracted from the samples. The cDNA synthesis, labeling, hybridization, microarray scanning and signal detection of the samples was carried out according to the instruction by Roche NimbleGen. Custom microarray slides containing 60-mer probes designed based on the public T. reesei genome sequence from http://genome.jgi-psf.org/Trire2/Trire2.home.html were used. The microarray data was analysed for differentially expressed protease genes with a statistical significance cut-off at P<0.01 by using the R packages Oligo, Limma and Mfuzz (http://www.bioconductor.org/).

Based on the microarray results, the expression of several protease genes was down-regulated in the low protease mutant 33SP #9. In addition to the previously identified proteases, altogether at least 18 additional proteases with clearly lowered expression were discovered.

The results obtained from the protein and RNA analysis are summarised in Table 2.

TABLE 2
Proteases being absent or less abundant in the culture
supernatants and/or having lower
expression level in the low protease mutants
compared to their parents. The proteases
were grouped according to the peptide
database MEROPS (http://merops.sanger.ac.uk/).
No, number of individual proteases
belonging to the group.

| | | Families represented |
|---|---|---|
| Protease Group (MEROPS) | No | (MEROPS) |
| Metallo Peptidase (M) | 11 | M1, M3, M6, M14, |
| | | M18, M28 |
| Serine Peptidase (S) | 7 | S1, S28, S8/S53 |
| Aspartic Peptidase (A) | 5 | A1 |
| Glutamic Peptidase (G) | 2 | G1 |
| Mixed peptidase (P) | 1 | P1 |

### Example 5. Genome Sequencing and Comparison

Genomic DNA was isolated from freeze-dried and ground mycelium of selected low protease strains with E.Z.N.A® SP Fungal DNA Mini Kit (Omega Bio-Tek Inc., USA) according to the manufacturer's instructions. The genomes were sequenced using the Illumina (Solexa) method and the draft genomes were assembled against the public Trichoderma reesei RutC-30 genome version 1.0 (TrireRUTC30_1) available from http://genome.jgi.doe.gov/TrireRUTC30_1/TrireRUTC30_1.home.html. The ID numbers derived from this genome are hereinafter referred to with a prefix Rut_. All differences in genomes against the public genome were analysed and the mutation profiles compared between the low protease strains. According to the genome sequencing, three individual low protease strains had mutations in the coding region of a predicted gene Rut_ID85889 (SEQ ID NO: 4-6). The corresponding gene in strain 33SP #9 (SEQ ID NO: 7) contained an insertion of two nucleotides inside the coding region of the predicted gene 840 bp downstream of the start codon. In strain 31UV #22 (SEQ ID NO: 9), the gene had a deletion of one nucleotide from the coding region 968 bp downstream of the start codon. According to the annotation of the gene Rut_ID85889, both the insertion and the deletion described above result in a frame-shift and formation of an early stop codon downstream of the mutations. The mutation in 31SP #4 (SEQ ID NO: 8) is a single point mutation 1224 bp downstream of the start codon resulting in the formation of an early stop codon.

For strain A21, the corresponding full-length gene Rut_ID85889 was PCR amplified from the A21 genomic DNA and sequenced directly from the PCR fragment using the ABI PRISM® 310 Genetic Analyzer by Applied Biosystems (Thermo Fisher Scientific Inc., USA). The nucleotide sequence of the corresponding gene in strain A21 (SEQ ID NO: 10) was found to contain a single point mutation 952 bp downstream of the start codon resulting in the formation of an early stop codon.

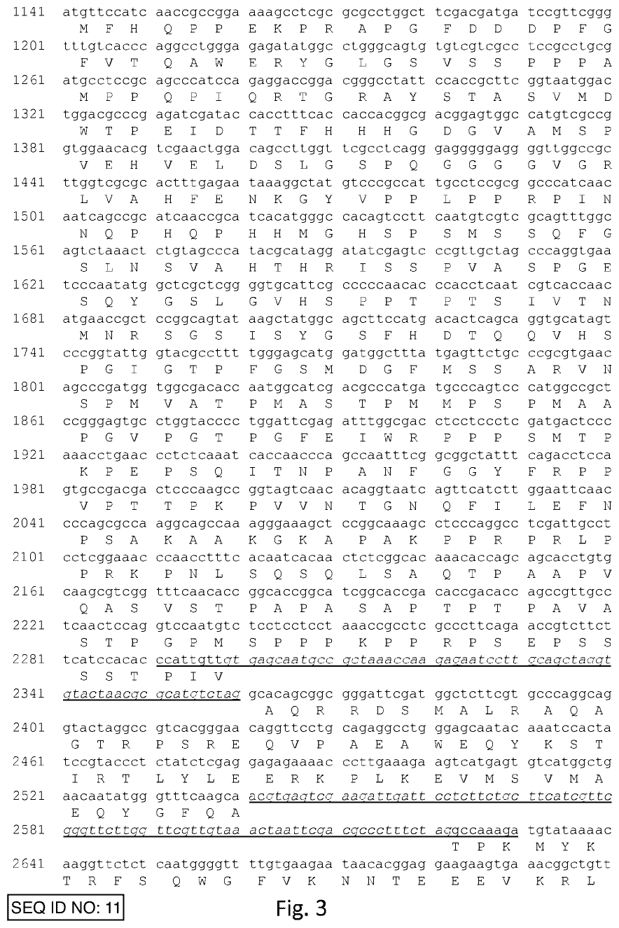

All of the mutations described above disrupt the full-length open reading frame of the Rut_ID85889 gene and the mutated genes, when translated, encode truncated protein products. The putative Rut_ID85889 was named as protease expression affecting gene, pea1. The pea1 gene in TrireRUTC30_1 genome is 2749 bp long including the stop codon and contains two introns, a 191 bp long intron 1029 bp downstream of the start codon and a 80 bp long intron 1402 bp downstream of the start codon. The annotation of the RutC-30 pea1 gene differs from the annotation of the gene in the corresponding genome region in the Trire2 genome, QM_ID123125 (SEQ ID NO: 1-3). The sequence of the hypothetical QM6a gene QM_ID123125 corresponds to the C-terminal nucleotide sequence of the Rut_ID85889. QM_ID123125 is 961 bp long and has a 42 bp intron 383 bp downstream of the start site (FIG. 2). Because of the discrepancies in the annotation of the Rut_ID85889 and QM_ID123125 genes, cDNA synthesis and sequencing of the pea1 cDNA was performed from a QM6a RNA sample (Example 6).

### Example 6. The pea1 Gene Annotation and Sequence Comparison

In order to confirm the nucleotide sequence of the pea1 gene and locus, a 4.7 kb fragment was PCR cloned using QM6a genomic DNA as template. The fragment was amplified using primers S-ppea1 (sense primer CGTTGGCTCGAGGCAACTGC (SEQ ID NO: 19)) and AS-3UTRout16 (anti-sense primer TGTCATCATGTCTTTATTCA (SEQ ID NO: 20)). The PCR reaction mixtures contained 1× Phusion HF buffer (Thermo Fisher Scientific Inc., USA), 0.23 mM dNTPs, 1.3 μM each primer and 1.3 units of Phusion High-Fidelity DNA polymerase (Thermo Fisher Scientific Inc., USA) per 50 μl reaction volume. The conditions for the PCR reactions were the following: 1 min initial denaturation at 98° C., followed by 29 cycles of 10 s at 98° C., 30 s annealing at 63° C., 1 min extension at 72° C. and a final extension at 72° C. for 5 min. The resulting 4.7 kb PCR fragment was cut from agarose gel and isolated using the QIAquick Gel Extraction Kit (Qiagen GmbH, Germany). The purified fragment was cloned into the PCR®4 Blunt-TOPOR Vector using the Zero Blunt® TOPO® PCR Cloning Kit (Thermo Fisher Scientific Inc., USA). The resulting plasmid was named pALK3535 and the Escherichia coli (TOP10) strain including the plasmid, RF11697, was deposited to the DSM collection under the accession number DSM32007. The PCR fragment in pALK3535 contains the full-length RutC-30 ID: 85889 gene and 1140 bp upstream and 821 bp downstream sequences (SEQ ID NO: 11). This fragment was sequenced using the ABI PRISM® 310 technology as described in Example 5. The sequence was identical to the nucleotide sequence in the public Trire2 and TrireRUTC30_1 genomes.

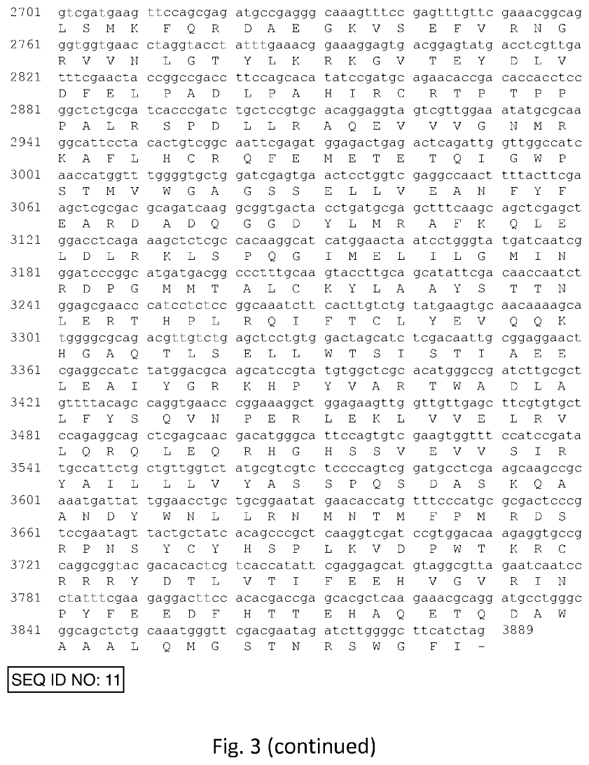

For the cDNA analysis, total RNA was isolated from deep frozen QM6a mycelium grown in cellulose inducing medium (Joutsjoki et al., 1993) with RNeasy® Plant Mini Kit (Qiagen GmbH, Germany) and mRNA translation to cDNA from the isolated RNA was done with Transcriptor First Strand cDNA Synthesis Kit (Roche Diagnostics GmbH, Germany) according to the manufacturer's instructions. The cDNA was PCR amplified using specific primers S-5UTR26 (sense primer CCAGAACAGCTCCGTCCTGG (SEQ ID NO: 21)) and AS-3UTRout16. The PCR reaction mixtures contained 1× Q5 Reaction buffer (New England Biolabs Inc., USA), 0.2 mM dNTPs, 0.5 μM each primer and 2 units of Q5® High-Fidelity DNA polymerase (New England Biolabs Inc., USA) and approximately 2 μl of cDNA per 50 μl reaction volume. The conditions for the PCR reactions were the following: 1 min initial denaturation at 98° C., followed by 31 cycles of 10 s at 98° C., 30 s annealing at 63° C., 1 min 20 s extension at 72° C. and a final extension at 72° C. for 4 min. The resulting 4.1 kb PCR fragment was cut and isolated from agarose gel. The purified fragment was cloned into the PCR®4 Blunt-TOPO® Vector using the Zero Blunt® TOPO® PCR Cloning Kit (Thermo Fisher Scientific Inc., USA). The resulting plasmid was named pALK3536 and the Escherichia coli (TOP10) strain including the plasmid, RF11698, was deposited to the DSM collection under the accession number DSM32008. The cDNA in pALK3536 includes 654 bps of the 5′UTR (untranslated region) and 821 bps of the 3′UTR (SEQ ID NO: 12). The fragment was sequenced and the sequence was compared to the corresponding pea1 gene cloned from QM6a (SEQ ID NO: 11). The results showed that the pea1 gene start and stop sites and the second intron were as predicted for the Rut_ID85889, but contrary to the Rut_ID85889 annotation, the first intron of pea1 is 62 bp long and located 1158 bp downstream of the start codon (FIG. 3).

The nucleotide sequence of the full-length pea 1 gene (SEQ ID NO: 11, nucleotides 1141-3889) and the deduced amino acid sequence (SEQ ID NO: 13) were used to search similar sequences from public sources. Searches were made using the FASTA search tools at the EMBL-EBI website by using the ENA sequence database for the nucleotide search (www.ebi.ac.uk/Tools/sss/fasta/nucleotide.html) and the UniProt Knowledgebase for the protein search (www.ebi.ac.uk/Tools/sss/fasta/). The searches were made using the default values. In addition, searches were done from available genome sequences of the strains belonging to Trichoderma genus. The Trichoderma genome sequences used in the searches were as follows: Trichoderma citrinoviride (http://genome.jgi.doe.gov/Trici1/Trici1.home.html), Trichoderma longibrachiatum (http://genome.jgi.doe.gov/Trilo1/Trilo1.home.html), Trichoderma virens (http://genome.jgi-psf.org/TriviGv29_8_2/TriviGv29_8_2.home.html), Trichoderma harzianum (http://genome.jgi.doe.gov/Triha1/Triha1.home.html), Trichoderma asperellum (http://genome.jgi.doe.gov/Trias1/Trias1.home.html), Trichoderma atroviride (http://genome.jgi.doe.gov/Triat2/Triat2.home.html). The identity values (%) to the most similar sequences identified from the searches were determined using the Pairwise Sequence Alignment tool at the EMBL-EBI website (for nucleotide sequences: www.ebi.ac.uk/Tools/psa/emboss_needle/nucleotide.html; for protein sequences: www.ebi.ac.uk/Tools/psa/emboss_needle/by using the default values (Gap open: 10 and Gap extend: 0.5). The results are shown in Tables 3A and 3B. The highest identities were to the homologous sequences from other Trichoderma species. The highest percentage of identity to a non-Trichoderma sequence was obtained with a hypothetical Ophiocordyceps sinensis OCS_06053 sequence, with 59.6% identity on the nucleotide level and 58.3% identity on the protein level.

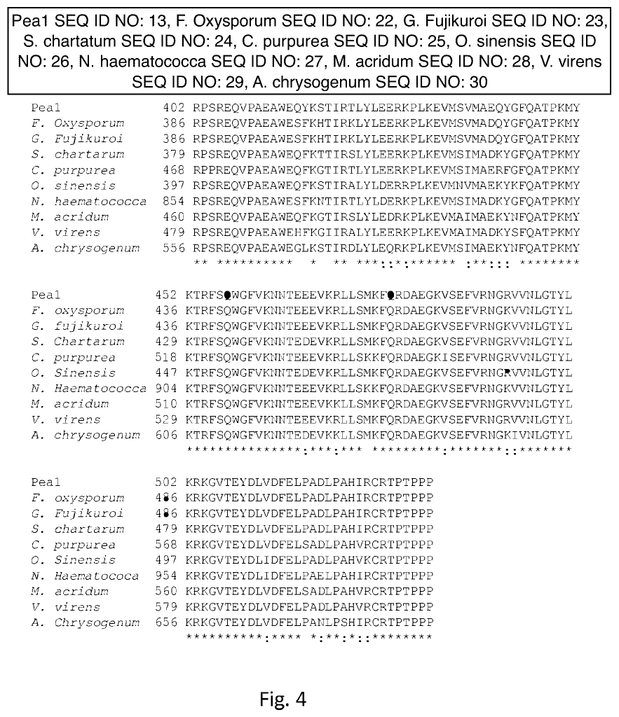

The Pea1 amino acid sequence was aligned with the homologous sequences obtained from other Trichoderma species and sequences having over 50% identity to the Pea1 protein, according to the FASTA protein search results. A highly conserved region was detected from the alignment. One sequence per genus was selected from the search results for further analysis. The identity between the Trichoderma species in the highly conserved Pea1 region, from Arg₄₀₂ to Pro₅₃₃ (132 residues), is at least 97% and similarity 99% whereas this region had at least 90% identity and 96% similarity to the sequences deriving from other filamentous fungal species, selected from the FASTA search results (Table 3C). Corresponding sequence regions were used in determining the degree of identity as shown in FIG. 4. Taxonomically (http://www.mycobank.org), all of the selected sequences originate from species belonging to the Sordariomycetes, subclass Hypocreomycetidae and order Hypocreales, indicating that this region is highly conserved in especially in Hypocreales. High values, 90.2% identity and 96.2% similarity were also found to e.g. Scedosporium apiospermum (SAPIO_CDS0483) sequence. The S. apiospermumspecies also belongs to the subclass Hypocreomycetidae, order Microascales.

The highly conserved Pea1 region contains a predicted pfam domain Clr5 (PF14420). The Clr5 domain is located at position Ala₄₁₀ to Lys₄₆₂ (53 residues) in Pea1 sequence. The Clr5 domain has been shown to be involved in silencing in fission yeast (Hansen, K. R., Hazan, I., Sreenath, S., Watt, S., Verhein-Hansen, J., Bähler, J., Martienssen R. A., Partridge, J. F., Cohen, A., Thon, G. 2011. H3K9me-independent gene silencing in fission yeast heterochromatin by Clr5 and histone deacetylases. PLOS Genetics 7, e1001268).

TABLE 3A
The identity values (%) obtained from Pairwise Sequence
Alignment of the nucleotide sequence
of full-length pea1 gene (SEQ ID NO: 11,
nucleotides 1141-3889). EMBOSS Needle
(EMBL-EBI, EMBOSS-Needle-Pairwise
Sequence Alignment, Matrix DNAfull,
Gap open 10, gap extend 0.5) at
www.ebi.ac.uk/Tools/psa/emboss_needle/
nucleotide.html was used for determining
the degree of identity.

| Name | Identity |
|---|---|
| Rut_ID85889 | 100 |
| Trichoderma citrinoviride ID:7704 (v1.0) | 90.0 |
| Trichoderma longibrachiatum ID:60713 (v1.0) | 89.7 |
| Trichoderma virens ID:58331 (v2.0) | 81.2 |
| Trichoderma harzianum ID:235354 (v1.0) | 80.6 |
| Trichoderma asperellum ID: 84188 (v1.0) | 76.9 |
| Trichoderma atroviride ID:280821 (v2.0) | 76.7 |
| Ophiocordyceps sinensis OCS_06053 | 59.6 |

TABLE 3B
The identity and similarity values (%) obtained from
Pairwise Sequence Alignment of the
full-length Pea1 amino acid sequence (SEQ
ID NO: 13, amino acids 1-868). EMBOSS
Needle (EMBL-EBI, EMBOSS-Needle-
Pairwise Sequence Alignment, Matrix
BLOSUM62, Gap open 10, gap extend 0.5) at
www.ebi.ac.uk/Tools/psa/emboss_needle/
was used for determining the degree of
identity and similarity.

| Name | Identity | Similarity |
|---|---|---|
| Rut_ID85889 | 95.0 | 95.0 |
| Trichoderma citrinoviride ID: 7704 (v1.0) | 96.0 | 96.0 |
| Trichoderma longibrachiatum | 91.5 | 93.9 |
| ID: 60713 (v1.0) | | |
| Trichoderma harzianum ID: 235354 (v1.0) | 88.3 | 93.3 |
| Trichoderma virens ID: 58331 (v2.0) | 85.9 | 90.7 |
| Trichoderma atroviride ID: 280821 (v2.0) | 83.0 | 89.6 |
| Trichoderma asperellum ID: 84188 (v1.0) | 82.9 | 90.1 |
| Ophiocordyceps sinensis OCS_06053 | 58.3 | 69.9 |

TABLE 3C
The identity and similarity values (%) obtained from
Pairwise Sequence Alignment of
the amino acid sequence of the Pea1 highly
conserved region (SEQ ID NO: 13,
amino acids 402-533) with the corresponding
region in other sequences. EMBOSS
Needle (EMBL-EBI, EMBOSS-Needle-
Pairwise Sequence Alignment, Matrix
BLOSUM62, Gap open 10, gap extend 0.5) at
www.ebi.ac.uk/Tools/psa/emboss_needle/
was used for determining the degree of
identity and similarity.

| Name | Identity | Similarity |
|---|---|---|
| Trichoderma citrinoviride ID: 7704 (v1.0) | 100 | 100 |
| Trichoderma longibrachiatum | 100 | 100 |
| ID: 60713 (v1.0) | | |
| Trichoderma atroviride ID: 280821 (v2.0) | 98.5 | 100 |
| Trichoderma asperellum ID: 84188 (v1.0) | 98.5 | 100 |
| Trichoderma harzianum ID: 235354 (v1.0) | 97.0 | 100 |
| Trichoderma virens ID: 58331 (v2.0) | 97.0 | 99.2 |
| Fusarium oxysporum FOVG_08585 | 95.5 | 97.7 |
| Gibberella fujikuroi FFUJ_12153 | 95.5 | 97.7 |
| Stachybotrys chartarum S40293_07230 | 94.7 | 100 |
| Claviceps purpurea CPUR_05697 | 92.4 | 97.0 |
| Ophiocordyceps sinensis OCS_06053 | 91.7 | 98.5 |
| Nectria haematococca | 91.7 | 98.5 |
| NECHADRAFT_85885 | | |
| Metarhizium acridum MAC_08836 | 91.7 | 97.7 |
| Villosiclava virens UV8b_6262 | 91.7 | 96.2 |
| Acremonium chrysogenum ACRE_079620 | 90.2 | 97.0 |

### Example 7. Construction of Cassettes for Deleting the Full-Length and Partial pea1 Gene from T. reesei

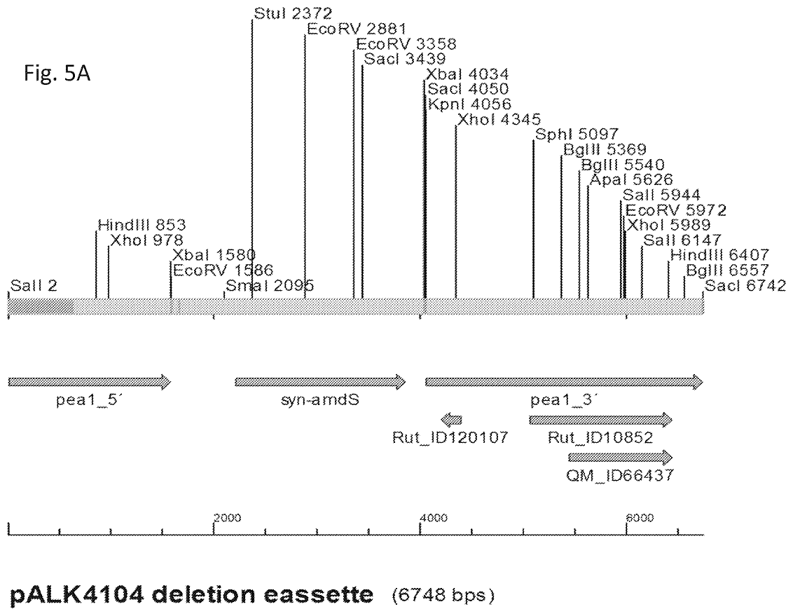

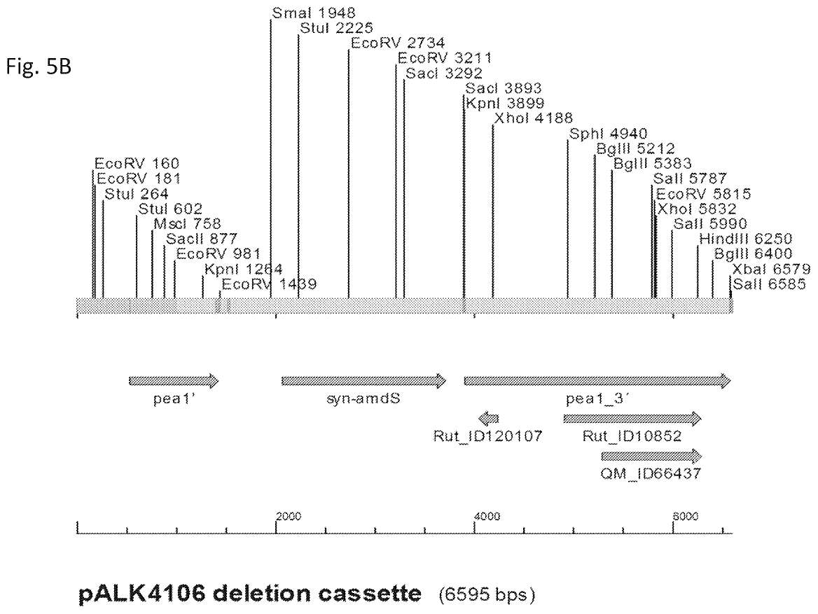

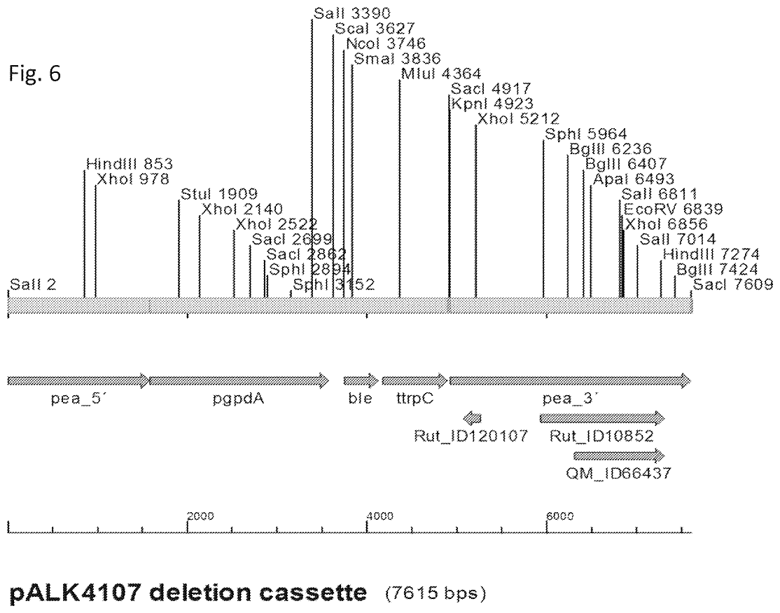

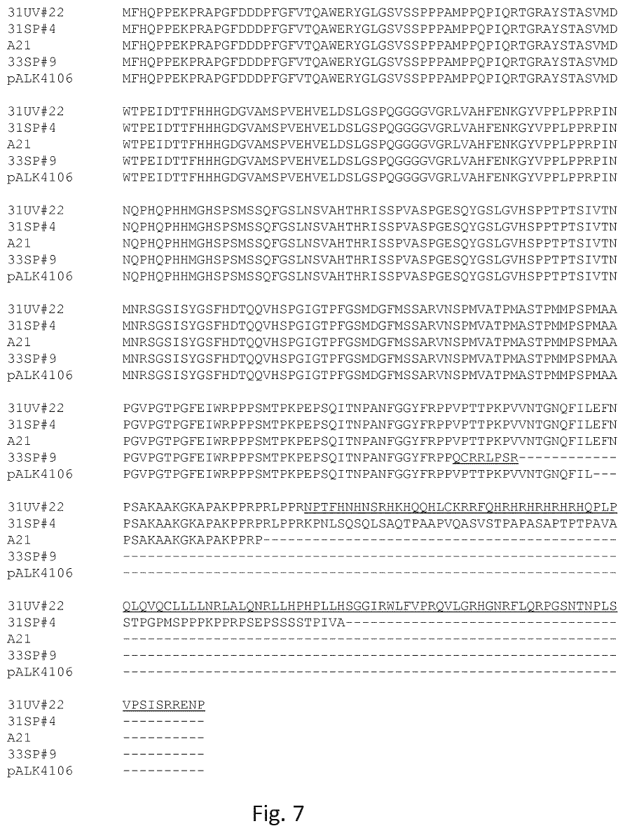

Altogether three deletion cassettes were planned and constructed, pALK4104 (FIG. 5A), pALK4106 (FIG. 5B) and pALK4107 (FIG. 6). The pALK4104 and pALK4107 were constructed for deleting the full-length pea1 gene and pALK4106 for partial pea1 deletion (truncation) from the genomes of T. reesei host strains. The length of the deduced amino acid sequence of the truncated Pea1 encoded by pALK4106 (297 amino acids) is in the range of the deduced Pea1 mutant protein in strains A21, 31SP #4, 33SP #9 and 31UV #22 (FIG. 7). All the cassettes contain a selection marker surrounded by flanking regions for targeting the cassette into an intended location in the T. reesei genome. For details, see below.

The pUC19 vector was used as a backbone in the plasmid constructions. The common molecular biology methods were used in enzyme treatments of DNA, PCR (polymerase chain reaction), E. coli transformations and isolation of plasmid DNA and DNA fragments for ligations and transformations. A genomic DNA preparation isolated from QM6a was used as a template in all the PCR reactions.

The pALK4104 deletion cassette contains:
A pea1 5′-flanking region for targeting the cassette into the pea1 locus for gene replacement, together with the 3′-flanking region (see below). The 5′-flanking region is the 1578 bp Sa/I-XbaI genomic fragment, the XbaI site locating 531 bp upstream from the pea1 gene start (first Met encoding ATG). The fragment was synthesized by PCR.
Synthetic amdS (acetamidase) encoding the acetamidase selection marker. A cDNA of the native Aspergillus nidulans amdS gene with additional modifications (deletion of chosen restriction sites) was used in the deletion cassette. The gene encodes the original AmdS amino acid sequence.
A pea1 3′-flanking region for targeting the cassette into the pea1 locus for gene replacement, together with the 5′-flanking region (see above). The 3′-flanking region is the 2676 bp KpnI-XbaI genomic fragment, the KpnI site locating 60 bp downstream from the pea1 gene's stop codon (TAG). This fragment was synthesized by PCR. It includes all the genes annotated into this region, according to both the public Trire2 or TrireRUTC30_1 genome sequences. The XbaI site at the 3′-end of the fragment is not available in the final construction due to filling in reaction (by Klenow fragment) done when constructing the plasmid.

The pALK4106 deletion cassette contains:
A pea1 5′-flanking region for targeting the cassette into the pea1 locus/gene for gene replacement, together with the 3′-flanking region (see below). The 5′-flanking region contains a partial pea1 promoter, starting immediately after the XbaI site in the promoter region (526 bps before the gene start codon, the XbaI site is not included) and ending immediately prior to the internal EcoRI site in the pea1 gene (892 bps from the gene start, the EcoRI site is not included). This fragment was synthesized by PCR. It encodes a truncated 297 amino acids Pea1 product (SEQ ID NO: 18).
Synthetic amdS (acetamidase) encoding the acetamidase selection marker. A cDNA of the native Aspergillus nidulans amdS gene with additional modifications (deletion of chosen restriction sites) was used in the deletion cassette. The gene encodes the original AmdS amino acid sequence.
A pea1 3′-flanking region for targeting the cassette into the pea1 locus for gene replacement, together with the 5′-flanking region (see above) was the same as the 3′-flanking fragment used in pALK4104 (see above).

The pALK4107 deletion cassette contains the identical 5′- and 3′-flanking regions to those included in pALK4104. The syn-amdS gene in pALK4104 (XbaI digestion of pALK4104, fill-in by Klenow) was replaced by the ble selection marker gene (with a promoter and terminator originating from Aspergillus nidulans) deriving from pAN8-1 (3313 bp Bg/II-XbaI fragment, the ends filled in using Klenow) and coding for phleomycin resistance (for more details, see the description of FIG. 6). The pALK4107 deletion cassette was used to delete the full-length pea1 gene from such T. reesei strains that already include the amdS marker gene, due to e.g. previous transformation of a gene expression cassette into the strain.

The 6756 bp pALK4104 and 6595 bp pALK4106 deletion cassettes for the T. reesei transformations were cleaved from the vector backbones by PstI-EcoRI digestions, were isolated from agarose gels and transformed (as described in Example 3) to protoplasts of a selection of T. reesei host strains, namely QM6a, RutC-30 and A33. The transformants were selected on acetamide plates and purified via single spores prior to streaking them on PD slants.

The transformations done using the pALK4107 deletion cassette are described in Example 9.

### Example 8. Characterisation of the pALK4104 and pALK4106 Transformant

The protease production of a selection of QM6a, RutC-30 and A33 transformants were analysed by growing the strains on skim milk plates. The host strains were used as controls. Transformants which produced lower amounts of protease in the plate assay compared to their host were found from each set of transformants (Table 4). The pea1 locus from the genomes of a selection of these transformants was analysed by Southern blot method. The pea1 gene was found to be deleted from the genomes of all the low protease pALK4104 transformants and truncated in the genomes of all the low protease pALK4106 transformants analysed by Southern blot. Strains with successful replacement of the pea1 gene with one copy (single-copy replacement) of the syn-amdS selection marker (in pALK4104 transformants) and replacement of the partial pea1 gene with the syn-amdS (in pALK4106 transformants, leading to truncation of the pea1 gene in these strains) were found from each set of transformants (Table 4).

TABLE 4
Summary on the pALK4104
(deletion of the full length pea1)
and pALK4106 (partial deletion/
truncation of pea1) transformants analysed on
skim milk plates and by Southern blot.
Amounts of low protease strains (reduced
halo compared to host) and single copy
(correct replacement) strains of all analysed
transformants are shown.

| | | | Single-copy |
|---|---|---|---|
| | | Low protease | replacement |
| | | transformants | strains |
| | Deletion cassette | (skim milk plate | (Southern blot |
| Host strain | transformed | assay) | analysis) |
| QM6a | pALK4104 | 4/18 | 3/4 |
| RutC-30 | ″ | 11/33 | 6/6 |
| A33 | ″ | 14/31 | 6/6 |
| QM6a | pALK4106 | 4/18 | 4/4 |
| RutC-30 | ″ | 10/29 | 4/6 |
| A33 | ″ | 12/30 | 6/6 |

Three single-copy replacement strains were chosen from each transformation and stored to RoaI culture collection. The low protease phenotype of these strains is further analysed by cultivating the transformants and their hosts (for comparison) in laboratory scale bioreactors. A cellulase inducing complex medium is used in the cultivations. The results are expected to correspond to those previously obtained from the cultivations of the low protease mutants (Example 2): the transformants with pea1 gene deletion and truncation produce lower protease activities compared to their hosts. The genetically modified strains with pea1 deletion or truncation are expected to produce similar or better amounts of secreted proteins and/or cellulase activities compared to their hosts as only the pea1 locus has been modified in these strains.

### Example 9. Deletion of pea1 from Strains Overproducing a Cellulase and a Laccase Enzyme

The deletion cassette pALK4107 for the T. reesei transformations was cleaved from the vector backbone by PstI-EcoRI digestion, was isolated from an agarose gel and transformed to protoplasts of two previously constructed strains producing recombinant enzymes. The strains transformed were as follows: RF5969 producing the 20K+CBD (expression from the pALK1769 cassette, Example 2) and RF5597 producing a laccase TaLcc1, originating from Thielavia arenaria (expression from the pALK1667 cassette, U.S. Pat. No. 7,927,849). In both cases, the gene encoding the recombinant enzyme was expressed using the strong native T. reesei cbh1 (cel7A) promoter. The transformation of the pALK4107 deletion cassette to RF5969 and RF5597 protoplasts was done as described in Example 3 but using phleomycin selection for screening of the transformants (Harkki et al., 1991). After purification via single spores, the transformants were streaked on PD slants.

The protease production of the transformants was analysed using skim milk plates (as explained in Example 1) using the transformation hosts as controls. Transformants producing lower amounts of proteases compared to their host were obtained from both the transformations.

The RF5969 transformants can be further tested on cellulase indicator plates containing e.g. Azo-CM-cellulose (Megazyme) and the RF5597 transformants on laccase indicator plates containing ABTS (Roche) to confirm the 20K+CBD and laccase production, respectively, of these strains. The transformation hosts are used as controls in the plate assays.

The chosen transformants with low protease production and confirmed production of the recombinant enzyme can be cultivated in shake flasks and/or bioreactors using cellulase inducing conditions. The lowered protease production compared to the hosts can be shown from the culture supernatants by activity assay(s). Increased production and better stability of the recombinant enzymes in the culture supernatant samples of the low protease strains compared to the hosts can be confirmed by known methods.

### Example 10. Characterisation of the Production Strains with pea1 Deletion

A set of RF5597 and RF5969 transformants which produced lower amounts of proteases compared to their hosts in the plate assay (Example 9) were further characterised. A Southern blot analysis confirmed that in all these strains the pea1 gene was replaced with the selection marker. The hosts and chosen transformants with confirmed deletion of the pea1 gene were cultivated in 0.5 L bioreactors using cellulase inducing conditions. The protease activity and other relevant enzyme activities were measured from the culture supernatants. The protease activity (HUT) was measured using haemoglobin substrate (as in Example 2). Cellulase activity (NCU, “neutral cellulase unit”) was analysed from RF5969 and its transformants. Carboxymethylcellulose (Sigma, low viscosity CMC) was used as a substrate in this analysis. The enzyme reaction was conducted at pH 7.0, 50° C. for 10 minutes and DNS method was used to measure the liberated reducing ends. As a soluble substrate was used in the analysis, there are no major differences in the specific activities between the 20K cellulase forms with and without the binding domain (CBD/CBM). The laccase activity was measured from RF5597 and its transformants at pH 4.5 using ABTS as a substrate (Niku-Paavola, M. L.; Karhunen, E.; Salola, P.; Raunio, V. (1988): Ligninolytic enzymes of the white-rot fungus Phlebia radiata. In Biochem. J. 254 (3), pp. 877-883).

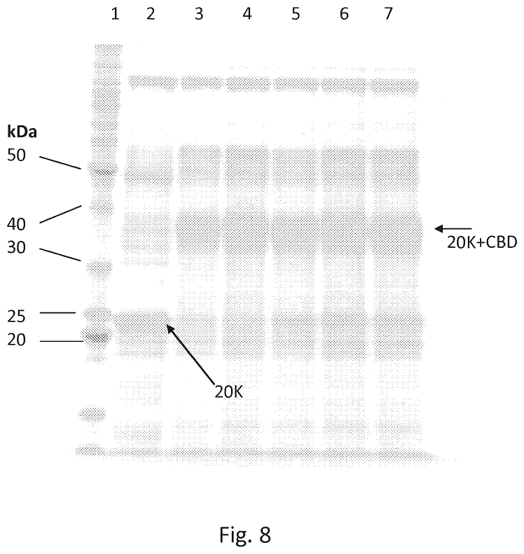

The RF5597 and RF5969 transformants with pea1 deletion produced clearly lower protease (HUT) activity compared to their hosts which have the wild type pea1 gene. The protease activities from the culture supernatants of the RF5597 transformants were, in average, only about 50% and of the RF5969 transformants, in average, only about 25% of the activity measured from the hosts culture supernatants. The cellulase (NCU) activity in the culture supernatants of the RF5969 transformants was increased up to 37% compared to the activity measured from the RF5969 cultivation. However, no increases in the laccase activities produced by the RF5597 transformants, compared to RF5597, were detected. To analyse the integrity and stability of the recombinant enzyme products, samples of the culture supernatants were run into SDS-PAGE gel. The TaLcc1 laccase protein band was similar (in mass and amount) from RF5597 and its transformants. However, there were clear differences in the recombinant cellulase protein produced by RF5969 and its transformants with pea1 deletion (FIG. 8). The major protein in the culture supernatant of RF5969 was not the full-length 20K+CBD but the 20K core form from which the CBD had been cleaved off. Only very minor amount of the full-length 20K+CBD was detectable in the gel. The RF5969 transformants with pea1 deletion produced mainly the full-length 20K+CBD and only very low relative amount of the 20K form. This result confirms that the pea1 deletion strains were able to produce higher amounts of the recombinant product and that the recombinant enzyme in the culture supernatants of the pea1 deletion strains was more stable than it was in the culture supernatant of the host.

The stabilities of the TaLcc1 products were further studied by incubating samples of culture supernatants at 30 and 50° C. (at pH 4) for up to three days. After the incubations samples were run into SDS-PAGE gel. The recombinant TaLcc1 was very stable in all the samples. However, after 3 days of incubation at 50° C. the TaLcc1 protein band was clearly more degraded in the culture supernatant of the host (RF5597) compared to the supernatants of the RF5597 Δpea1 transformants. This result further confirms the increased stability of the products obtained from the strains with a non-functional pea1 gene.

Similar results were obtained from RF5597 and RF5969 transformants from which the pea1 gene was deleted using the pALK4116 deletion cassette. In this cassette, the ble marker gene in pALK4107 was replaced by the hph marker gene encoding resistance for hygromycin B (Mach, R. L.; Schindler, M.; Kubicek, C. P. (1994): Transformation of Trichoderma reesei based on hygromycin B resistance using homologous expression signals. In Curr. Genet. 25 (6), pp. 567-570).

### Example 11. Disruption of pea1 Homologue from Fusarium Species

Many fungal species contain a homologue of the T. reesei pea1 gene, as described in Example 6. The encoded full-length T. reesei Pea1 homologues from Fusarium oxysporum, e.g. FOVG_08585 and FOZG_02804 (amino acids 1-887) have identity values of 57.2 and 57.1% and similarity values of 68.8 and 68.7%, respectively, to the full-length T. reesei Pea1 (SEQ ID NO:13, amino acids 1-868; alignment done using EMBL-EBI, EMBOSS-Needle—Pairwise Sequence Alignment, Matrix BLOSUM62, Gap open 10, gap extend 0.5 at www.ebi.ac.uk/Tools/psa/emboss_needle/). The corresponding identity and similarity values between the T. reesei Pea1 and the full-length Fusarium (Gibberella) fujikuroi Pea1 homologue (e.g. FFUJ_12153, amino acids 1-882) are 57.5 and 68.9%, respectively. The deduced amino acid sequences of the full-length Pea1 homologues from the F. oxysporum and F. fujikuroi are highly similar with each other, the identity and similarity values between the above full-length amino acid sequences being 96.6 and 97.4%, respectively.

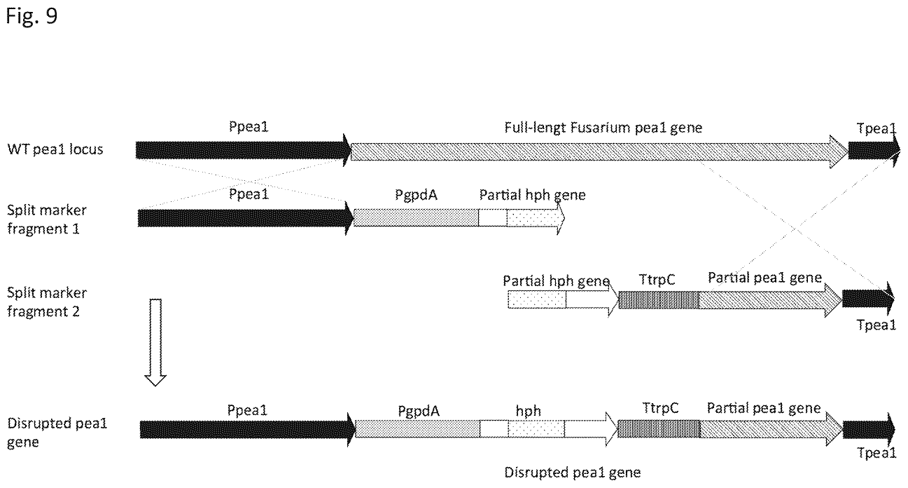

To confirm that the role of the Pea1 homologues in other fungi is similar to that in T. reesei, a split marker approach (FIG. 9) was designed to disrupt the pea1 homologues from two Fusarium species, F. oxysporum and F. fujikuroi. The ˜3 kb split marker fragment 1 contained a promoter region of the F. oxysporum Fo47 pea1 gene (1468 bp, nts from −1483 to −16 from the start codon, to target the fragment to pea1 locus) and the 5′ half of the hph marker gene (from nucleotide 1 to 615 and the Aspergillus gpdA promoter). The ˜3 kb split marker fragment 2 contained the 3′ half of the hph selection marker (from nucleotide 166 to 1026 and the Aspergillus trpC terminator region) and partial F. oxysporum pea1 gene and its terminator region (1358 bp; starting from the nt 1667 of the gene and ending 380 nts after the pea1 stop codon, to target the fragment to pea1 locus). Thus, both the split marker fragments included the same 450 bp middle part of the hph gene. When the two split marker fragments are transformed into the same host, they recombine with the corresponding pea1 regions in the genome. When they also recombine with each other at the common middle part region of hph, the selection marker becomes functional. Using the designed approach, a functional selection marker in the transformants was expected to be linked to a disrupted pea1 gene at high frequency.

As the sequences of the F. oxysporum and F. fujikuroi pea1 genes and their 5′- and 3′-regions are highly similar (but not identical) with each other, the same split marker fragments were used for disruption of the pea1 genes from both the species.

### Example 12. Transformation of Fusarium oxysporum and F. fujikuroi and Analysis of the Transformants

Fusarium oxysporum Fo47 and F. fujikuroi IMI58289 strains were transformed using the designed and synthesized split marker fragments (Example 11). The method described in Wiemann et al. (PLos Pathog. 2013; 9(6):e1003475 and references within) was used in the fungal transformations. Altogether 96 F. oxysporum and 46 F. fujikuroi transformants were obtained. The targeted DNA modification (disruption of the pea1 homologue) was analysed from 20 F. oxysporum and 10 F. fujikuroi transformants using diagnostic PCR. The primers in the PCR reaction were designed from the end of the pea1 5′-flank in the split marker fragment 1 (from the pea1 promoter, nucleotides from −38 to −21 from the ATG) and the beginning of the 3′-flank in the split marker fragment 2 (nucleotides 1716-1695 of the F. oxysporum pea1 gene). The designed diagnostic PCR reaction results to a 1.6 kb fragment from the native (complete) Fusarium pea1 gene whereas the length of the product from a disrupted gene is 2.5 kb.

From most of the transformants a sole 2.5 kb PCR product was obtained indicating a successful integration of the full-length marker into the pea1 locus and disruption of the pea1 gene. The pea1 flanking fragments from F. oxysporum could be used for disruption of the pea1 from both the Fusarium species.

A selection of transformants were purified which were shown by diagnostic PCR to contain a disrupted pea1 gene. Four transformants from each species and their parent strains were cultivated in shake flasks on casein-based induction medium (FusP) with and without supplementation of 0.5 g/L of CasAmino acids. The FusP medium contained (per 1000 ml): 20 ml of 50×FusP salts (26 g/L KCl, 82 g/L K₂HPO₄, 43 g/L NaH₂PO₄xH₂O, pH adjusted to 7.5 using NaOH), 10 g/L glucose, 5 g/L casein (Sigma C8654), 2 ml of 1 M MgSO₄, 1 ml of 1000×trace elements solution (contains, per 100 ml: 2.2 g ZnSO₄x7H₂O, 1.1 g H₃BO₃, 0.5 g MnCl₂x4H₂O, 0.5 g FeSO₄x7H₂O, 0.17 g CoCl₂x6H₂O, 0.16 g CuSO₄x5H₂O, 0.15 g Na₂MoO₄x2H₂O, 5.0 g Na₂EDTAx2H₂O, pH adjusted to 6.5 using KOH). Interestingly, the transformants with the disrupted pea1 gene showed hardly any growth on the medium which was not supplemented with the CasAmino acids, indicating that these strains were unable to use casein as a nitrogen source. All the strains, however, grew well in the medium supplemented with the CasAmino acids. Samples were taken from these cultures after 6 days of cultivation at 25° C. Extracellular proteolytic activities were measured from the culture supernatants based on a procedure described by Holm (Holm K.A. 1980. Automated colorimetric determination of acid proteinase activity in fermentation samples using a trinitrobenzenesulphonic acid reagent. Analyst. 105(1246):18-24).

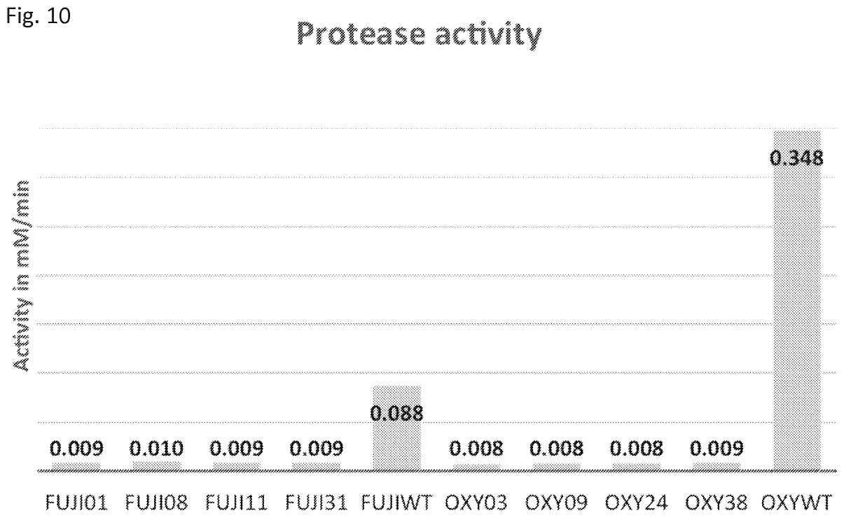

The protease activities determined from the culture supernatants of all the eight transformants with disrupted pea1 gene were very low compared to the activities from the culture supernatants of the parent strains (FIG. 10). The protease activity in the culture supernatants of the F. fujikuroi transformants was about 10-fold lower than that in the culture supernatant of the parent strain. The protease activity measured from the culture supernatants of the F. oxysporum transformants was about 40-fold lower than that from the parent strain.

The disruption of the pea1 homologue from Fusarium species was successful with the method used. The Fusarium transformants with disrupted pea1 show a distinct protease-deficient phenotype, like that of Trichoderma strains with non-functional pea1 gene.

The results show that the Trichoderma reesei low protease strains lacking functional pea1 give benefits when used as hosts for production of proteins, and especially protease sensitive proteins. At least similar, or in several cases even higher production yields of proteins can be reached with these strains compared to the yields obtained when the parents of these strains are used as hosts for the same enzyme products. In addition, the enzyme products obtained from the strains lacking a functional pea1 are more stable compared to the corresponding products from the parents of these strains.

The pea1 homologues can be found in the genome of several fungal species. Our results show that disruption of the pea1 homologues from species other than T. reesei lead to similar protease deficient phenotypes as shown for the T. reesei strains which lack a functional pea1. The results confirm the role of pea1 and its homologues as important factors for affecting protease expression. Significant improvements in protein yields and stability of products can be achieved by disrupting the pea1 from the production strains of different species.

The foregoing description has provided, by way of non-limiting examples of particular implementations and embodiments of the invention, a full and informative description of the best mode presently contemplated by the inventors for carrying out the invention. It is however clear to a person skilled in the art that the invention is not restricted to details of the embodiments presented in the foregoing, but that it can be implemented in other embodiments using equivalent means or in different combinations of embodiments without deviating from the characteristics of the invention.

Furthermore, some of the features of the afore-disclosed embodiments of this invention may be used to advantage without the corresponding use of other features. As such, the foregoing description shall be considered as merely illustrative of the principles of the present invention, and not in limitation thereof. Hence, the scope of the invention is only restricted by the appended patent claims.

1. A protease deficient host cell comprising at least one inactivation in a chromosomal gene comprising a nucleic acid sequence encoding a polypeptide having at least 90% sequence identity with the amino acids 402-533 of SEQ ID NO: 13, wherein the protease deficient host cell has a lower expression level of aspartic proteases, serine proteases, glutamic proteases, and metalloproteases, compared to the expression level of aspartic proteases, serine proteases, glutamic proteases, and metalloproteases of a corresponding host cell in which the chromosomal gene is not inactivated.

2. The protease deficient host cell of claim 1, wherein the protease deficient host cell has a lower expression level of at least one protease belonging to metallo peptidases, serine peptidases, aspartic peptidases, glutamic peptidases, or mixed peptidases.

3. The protease deficient host cell of claim 1, wherein the protease deficient host cell has a lower expression level of at least one protease in the protease families M1, M3, M6, M14, M18, M28, S1, S28, S8, S53, A1, G1, and P1.

4. The protease deficient host cell of claim 1, wherein the inactivated chromosomal gene is inactivated by disruption.

5. The protease deficient host cell of claim 1, wherein the protease deficient host cell has a reduced protease activity compared to a wild-type protease deficient host cell without said inactivation.

6. The protease deficient host cell of claim 5, wherein the reduced protease activity is affected by the inactivation in the chromosomal gene comprising a nucleic acid sequence encoding a polypeptide having at least 90% sequence identity with the amino acids 402-533 of SEQ ID NO: 13.

7. The protease deficient host cell of claim 1, wherein the inactivation is carried out by disruption; inhibition of translation or transcription of a chromosomal gene; at least partial deletion, mutation, insertion, or silencing; by RNA interference, or by CRISPR/Cas9 technology.

8. A protein preparation comprising secreted recombinant protein produced by the protease deficient host cell of claim 1, and a stabilizer.

9. The protein preparation of claim 8 further comprising at least one of preservative, fragrant, buffer, salt, and colorant.

10. A protein preparation comprising the protease deficient host cell of claim 1.

11. The protein preparation of claim 8 comprising non-degraded secreted recombinant protein having an increased stability compared to secreted recombinant protein produced in the same host cell without said inactivation.
